(19) United States
(12) Patent Application Publication
(10) Pub. No.: US 2016/0279079 A1
(43) Pub. Date: Sep. 29, 2016

(54) COMPOSITIONS AND METHODS FOR TREATING BONE DISEASES

(71) Applicant: INDIANA UNIVERSITY RESEARCH AND TECHNOLOGY CORPORATION, Indianapolis, IN (US)

(72) Inventors: Hiroki YOKOTA, Carmel, IN (US); Kazunori HAMAMURA, Indianapolis, IN (US); Ping ZHANG, Indianapolis, IN (US)

(21) Appl. No.: 14/778,181

(22) PCT Filed: Mar. 7, 2014
(86) PCT No.: PCT/US2014/021682

§ 371 Date: Sep. 18, 2015

Related U.S. Application Data
(60) Provisional application No. 61/803,223, filed on Mar. 19, 2013.

(51) Int. Cl. A61K 31/155; A61K 31/47

(52) U.S. Cl. CPC A61K 31/155; A61K 31/47

(57) ABSTRACT

Described herein are methods for treating bone diseases or defects. The methods include administering to a host animal therapeutically effective amounts of one or more compounds that are selective inhibitors of dephosphorylation of eIF2α.

### GOVERNMENT RIGHTS

This invention was made with government support under DOD W8IXWH-11-1-0716 awarded by the Department of Defense. The government has certain rights in the invention.

### TECHNICAL FIELD

The invention described herein pertains to the treatment of bone diseases. In particular, the invention described herein pertains to the treatment of bone diseases responsive to the inhibition of osteoclastogenesis and/or stimulation of osteoblast development and finction.

### BACKGROUND AND SUMMARY OF THE INVENTION

Osteoblasts and osteoclasts are the two major types of bone cells in bone remodeling. Osteoblasts are bone-forming cells originated from mesenchymal stem cells, while osteoclasts are bone-resorbing cells derived from hematopoietic stem cells. These two types of cells orchestrate a complex remodeling process, in which mineralized bone matrix is degraded by osteoclasts and newly formed by osteoblasts [1, 2]. In order to maintain proper bone mass, exercise and calcium rich diets are recommended. However, a failure of the coordinated action such as in osteoporosis, which is a common form of bone loss prevailing among postmenopausal women, increases risk of bone fracture [3]. In order to develop therapeutic drugs for treatment of osteoporosis, an understanding of signaling pathways that govern osteoclastogenesis—development of pre-osteoclasts (monocyte/macrophage) to multi-nucleated osteoclasts—is required.

Currently, the most common medications, prescribed for preventing bone loss in patients with osteoporosis, are bisphosphonates. Bisphosphonates preferentially bind to calcium in bone and induce apoptosis of osteoclasts [15]. Other medications using neutralizing antibodies targeted to RANKL would block osteoclastogenesis by mimicking OPG's binding to RANKL [16]. RANKL is a cytokine belonging to the tumor necrosis factor family, and is involved in T cell-dependent immune responses as well as differentiation and activation of osteoclasts [9, 10].

A novel target for treating such diseases is the protein complex, eIF2, which is a heterotrimer essential for protein synthesis, and eIF2α is one of its major components together with eIF2β and eIF2γ [4]. In response to various stresses such as oxidation, radiation, and stress to the endoplasmic reticulum that potentially lead to cellular apoptosis, a serine residue of eIF2α is phosphorylated. This action would initiate a pro-survival program by lowering general translation efficiency except for a group of genes that includes activating transcription factor 4 (ATF4) [5]. ATF4 is a transcription factor critical for osteoblastogenesis and bone formation [6]. In osteoblasts elevation of phosphorylated eIF2α (p-eIF2α) is reported to stimulate the expression of ATF4 [7, 8]. Little is known, however, about potential effects of p-eIF2α on development of osteoclasts.

In response to various stresses including viral infection, nutrient deprivation, and stress to the endoplasmic reticulum, eukaryotic translation initiation factor 2 alpha (eIF2α) is phosphorylated to cope with stress induced apoptosis. Although bone cells are sensitive to environmental stresses that alter the phosphorylation level of eIF2α, little is known about the role of eIF2α mediated signaling during the development of bone-resorbing osteoclasts. It has been discovered herein that by selectively inhibiting de-phosphorylation of eIF2α, the effects of phosphorylation of eIF2α on osteoclastogenesis of RAW264.7 pre-osteoclasts as well as development of MC3T3 E1 osteoblast-like cells are affected. Two illustrative agents, salubrinal and guanabenz, demonstrate that selectively inhibiting de-phosphorylation of eIF2α, results in stimulated matrix deposition of osteoblasts through upregulation of activating transcription factor 4 (ATF4), reduced expression of nuclear factor of activated T cells cl (NFATcl) and inhibited differentiation of RAW264.7 cells to multi-nucleated osteoclasts. In contrast, partial silencing of eIF2α with RNA interference reduced suppression of salubrinal/guanabenz-driven downregulation of NFATc 1. It has been observed herein that the elevated phosphorylation level of eIF2α not only stimulates osteoblastogenesis but also inhibits osteoclastogenesis through regulation of ATF4 and NFATc1, supporting the role of such selective inhibitor of de-phosphorylation of eIF2α in treating and/or preventing bone loss in osteoporosis, fracture, and other bone diseases.

It has also been discovered herein that the compounds described herein can regulate expression of NFATcl at a transcriptional level. Without being bound by theory, it is believed herein that the elevation of p-eIF2α stimulates osteocalcin expression through upregulation of ATF4 in osteoblasts and inhibits TRAP expression via downregulation of NFATcl in pre-osteoclasts. Silencing eIF2α with RNA interference reduces suppression of salubrinal/guanabenz-driven downregulation of NFATcl. Thus, the compounds described herein are useful in regulating bone remodeling through eIF2α-mediated signaling for combating bone loss in osteoporosis, and related diseases.

Osteoblasts and osteoclasts extensively interact through molecular pathways including RANK (receptor activator of nuclear factor kappa-B)/RANKL (RANK ligand)/OPG (osteoprotegerin) signaling [9, 10] and Wnt signaling [11]. It has been discovered herein that osteoclastogenesis is regulated by signaling molecules that also affect osteoblastogenesis. Furthermore, it has been discovered that osteoclastogenesis is influenced by various stresses such as estrogen deficiency and disuse or unloading [12]. It has also been discovered herein that elevation of p-eIF2α suppresses differentiation of pre-osteoclasts to multi-nucleated osteoclasts, and that elevation of p-eIF2α can provide stress-relieving effects on osteoblasts.

In one embodiment, described herein are compounds, compositions, methods, and uses for treating and/or preventing bone loss in osteoporosis, fracture, and other bone diseases. The compounds, compositions, methods, and uses include selective inhibitors de-phosphorylation of eIF2α, such as salubrinal and guanabenz [13, 14]. It is understood herein that the signaling pathway, mediated by eIF2α, is not directly linked to known agents for osteoclastogenesis such as calcium binding agents and RANKL.

In another embodiment, pharmaceutical compositions containing one or more of the compounds are also described herein. It is to be understood that the compositions may include other component and/or ingredients, including, but not limited to, other therapeutically active compounds, and/or one or more carriers, diluents, excipients, and the like.

It is appreciated herein that the compounds described herein may be used alone or in combination with other compounds useful for treating bone diseases, including those compounds that may be therapeutically effective by the same or different modes of action. In addition, it is appreciated herein that the compounds described herein may be used in combination with other compounds that are administered to treat other symptoms of bone disease.

### BRIEF DESCRIPTION OF THE DRAWINGS

FIG. 1. Osteogenic effects of salubrinal on MC3T3 E1 (clone 14) osteoblast cells. CN=control, Sal=salubrinal, and Tg=thapsigargin. The double asterisk indicates p<0.01 in comparison to CN. (A) Cell mortality ratio and relative cell numbers. (B) No activation of cleaved caspase 3 by salubrinal. (C) Alizarin red S staining area in response to 5, 10, and 20 μM salubrinal.

FIG. 2. Upregulation of p-eIF2α, ATF4 and osteocalcin by salubrinal in MC3T3 E1 (clone 14) osteoblast cells in response to 5 μM salubrinal. CN=control, Sal=salubrinal, and NC=non-specific control siRNA. The double asterisk indicates p<0.01 in comparison to CN or NC. The double dagger indicates with p<0.01 in comparison to the salubrinal-treated NC siRNA cells. (A) Western blot analysis of p-eIF2α and ATF4. (B) Salubrinal driven elevation of osteocalcin mRNA level. (C) ATF4 level after transfecting siRNA specific to ATF4. (D) Relative mRNA levels of ATF4 in response to RNA interference with ATF4 siRNA and non-specific control (NC) siRNA. (E) Relative mRNA levels of osteocalcin (OCN). The asterisk is for the comparison to the control with NC siRNA, and the dagger is the comparison between the samples transfected with ATF4 siRNA.

FIG. 3. Inhibitory effects of salubrinal on RAW264.7 pre-osteoclasts. CN=control, and Sal=salubrinal. The single and double asterisks indicate p<0.05 and p<0.01 in comparison to the RANKL-treated cells, respectively. (A) Cell mortality ratio. (B) Relative cell numbers. (C) Dose-dependent suppression of RANKL driven activation of osteoclasts by salubrinal. (D) Dose-dependent suppression of TRAP-positive multi-nucleated cells by salubrinal.

FIG. 4. Reduction of RANKL-induced NFATcl expression by salubrinal. Sal=salubrinal. The relative intensity of NFATcl to β-actin is shown. (A) Expression of NFATcl (2 days after RANKL administration). (B) Expression of NFATcl (4 days after RANKL administration).

FIG. 5. Effects of salubrinal on mRNA expression levels of NFATcl, c-Fos, TRAP, and OSCAR. CN=control. The single and double asterisks indicate significant decreases with p<0.05 and p<0.01 in comparison to the RANKL-treated cells, respectively. The single and double daggers indicate significant increases with p<0.05 and p<0.01 in comparison to the RANKL-treated cells, respectively. (A) Messenger RNA levels (2 days after RANKL administration). (B) Messenger RNA levels (4 days after RANKL administration).

FIG. 6. Temporal expression profile of p-ERK, p-p38 MAPK, p-NFicB, p-eIF2α and NFATcl in the presence and absence of 20 μM salubrinal. (A) Western blot analysis of p-ERK, p-p38 MAPK, p-NFκB, and p-eIF2α at 15, 30, 60 and 120 min. (B) Western blot analysis of eIF2α-p and NFATcl. (C) Comparison of the expression level of eIF2α-p and NFATcl with and without 20 μM salubrinal. The normalized level of “1” was defined as the level for the cells that were not treated with RANKL without administration of salubrinal.

FIG. 7. Inhibitory effects of guanabenz on development of RAW264.7 pre-osteoclasts. CN=control, and Gu=guanabenz. The single and double asterisks indicate significant decreases with p<0.05 and p<0.01 in comparison to the RANKL-treated cells, respectively. (A) Cell mortality ratio. (B) Relative cell numbers. (C) Dose-dependent suppression of RANKL driven activation of osteoclasts by guanabenz. (D) Dose-dependent suppression of TRAP-positive multi-nucleated cells by guanabenz.

FIG. 8. Reduction of RANKL-induced NFATcl expression by guanabenz. CN=control, and Gu=guanabenz. The single and double asterisks indicate significant decreases with p<0.05 and p<0.01 in comparison to the RANKL-treated cells, respectively. (A) Expression of NFATcl (2 days after RANKL administration). (B) Messenger RNA levels of NFATcl, c-Fos, TRAP, and OSCAR (2 days after RANKL administration).

FIG. 9. Temporal expression profile of p-eIF2α and NFATcl in the presence and absence of 20 μM guanabenz. Gu=guanabenz. (A) Western blot analysis of eIF2α-p and NFATcl. (B) Comparison of the expression level of eIF2α-p and NFATcl with and without 20 μM guanabenz. The normalized level of “1” was defined as the level for the cells that were not treated with RANKL without administration of guanabenz.

FIG. 10. Reduction in salubrinal/guanabenz driven suppression of NFATcl expression by RNA interference specific for eIF2α. Sal=salubrinal, Gu=guanabenz, and NC=non-specific control siRNA. The single and double asterisks indicate significant changes to the RANKL-treated NC siRNA cells with p<0.05 and p<0.01, respectively. The single and double daggers indicate significant changes to the salubrinal or guanabenz-treated NC siRNA cells with p<0.05 and p<0.01, respectively. (A) eIF2α level after transfecting siRNA specific to eIF2α. (B) Western blot analysis of p-NFκB and NFATcl. (C) Comparison of the expression level of NFATcl between control siRNA and eIF2α siRNA.

FIG. 11 shows, though without being bound by theory, an illustrative mechanism of eIF2α signaling on osteoclastogenesis through NFATcl.

FIG. S1. Osteogenic effects of salubrinal on MC3T3 E1 osteoblast cells. CN=control, and Sal=salubrinal. The double asterisk indicates p<0.01 in comparison to CN. (A) Cell mortality ratio in response to 0.1-30 μM salubrinal. (B) Relative cell numbers in response to 0.1-30 μM salubrinal. (C) Alizarin red S staining area in response to 0.1, 0.5, 1, 5, 10, and 20 μM salubrinal.

FIG S2. Upregulation of p-eIF2α, ATF4 and osteocalcin by salubrinal in MC3T3 E1 osteoblast cells. CN=control, and Sal=salubrinal. The single and double asterisks indicate p<0.05 and p<0.01 in comparison to CN, respectively. (A) Western blot analysis of p-eIF2α and ATF4. (B) Salubrinal-driven elevation of the levels of ATF4 mRNA and osteocalcin mRNA.

FIG. S3. Osteogenic effects of guanabenz on MC3T3 E1 osteoblast cells. CN=control, and Gu=guanabenz. The single and double asterisks indicate p<0.05 and p<0.01 in comparison to CN, respectively. (A) Cell mortality ratio. (B) Relative cell numbers. (C) Western blot analysis of p-eIF2α and ATF4. (D) Guanabenz-driven elevation of the osteocalcin mRNA level.

FIG. S4. No significant effects of salubrinal and guanabenz on cell mortality, cell numbers, and expression of NFATcl and TRAP in RAW264.7 cells in the absence of RANKL administration. CN=control, Sal=salubrinal, and Gu=guanabenz. (A) Cell mortality ratio. (B) Relative cell numbers. (C) Expression of NFATcl. (D) Relative mRNA levels of NFATcl and TRAP.

### DETAILED DESCRIPTION

In one embodiment, selective inhibitors of de-phosphorylation of eIF2α are described herein for treating and/or preventing bone loss in osteoporosis, fracture, and other bone diseases. Illustrative inhibitors include compounds of the formula

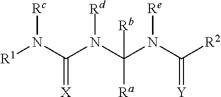

or a pharmaceutically acceptable salt thereof, wherein:

X is O or S;

Y is O or S;

R¹ is alkyl, cycloalkyl, alkenyl, cycloalkenyl, aryl, or heteroaryl, each of which is optionally substituted;

R² is alkyl, cycloalkyl, alkenyl, cycloalkenyl, aryl, heteroaryl, cycloalkylalkyl, cycloalkenylalkyl, arylalkyl, heteroarylalkyl, cycloalkylalkenyl, cycloalkenylalkenyl, arylalkenyl, or heteroarylalkenyl, each of which is optionally substituted;

R^{a} is optionally substituted alkyl;

R^{b} is H or optionally substituted C₁-C₆ alkyl;

R^{c}, R^{d}, and R^{e} are each independently selected from the group consisting of H, optionally substituted C₁-C₆ alkyl, acyl, or a prodrug capable of releasing the attached nitrogen in vivo.

It is to be understood that the various genera and subgenera of each of X, Y, R¹, R², R^{a}, Rb, R^{c}, R^{d}, and R^{e} are described herein. It is to be understood that all possible combinations of the various genera and subgenera of each of X, Y, R¹, R², R^{a}, Rb, R^{c}, R^{d}, and R^{e} are described herein represent additional illustrative embodiments of compounds of the invention described herein. It is to be further understood that each of those additional illustrative embodiments of compounds may be used in any of the compositions, methods, and/or uses described herein.

Further illustrative inhibitors include compounds of the formula

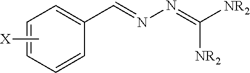

where

X represents five substituents each independently selected from hydrogen, halo, hydroxy, amino, thio, carboxylate or a derivative thereof, sulfinyl or a derivative thereof, sulfonyl or a derivative thereof, phosphinyl or a derivative thereof, or phosphoryl or a derivative thereof, or alkyl, alkenyl, alkynyl, cycloalkyl, cycloalkenyl, heteroalkyl, heteroalkenyl, cycloheteroalkyl, cycloheteroalkenyl, aryl, heteroaryl, arylalkyl, or heteroarylalkyl, each of which is optionally substituted; and

R is independently selected in each instance from hydrogen, hydroxy, amino, carboxylate and derivatives thereof, sulfonyl and derivatives thereof, and alkyl, alkenyl, alkynyl, cycloalkyl, cycloalkenyl, heteroalkyl, heteroalkenyl, cycloheteroalkyl, cycloheteroalkenyl, aryl, heteroaryl, arylalkyl, and heteroarylalkyl, each of which is optionally substituted, and nitrogen prodrug forming groups.

MC3T3 E1 osteoblast-like cells [17] and RAW264.7 cells [18] are employed to evaluate osteoblastogenesis and osteoclastogenesis, respectively. In the presence and absence of salubrinal and guanabenz, MC3T3 E1 cells are cultured in an osteogenic medium for evaluation of matrix deposition, while RAW264.7 cells in an osteoclast differentiation medium for evaluation of multi-nucleation. Alizarin Red S staining is performed to evaluate osteoblast mineralization for MC3T3 E1 cells, and TRAP staining is conducted to determine multi-nucleated osteoclasts proliferation for RAW264.7 cells. To analyze molecular signaling pathways, quantitative real-time PCR and Western blot analysis are conducted. The mRNA levels of ATF4, osteocalcin, c-Fos [19], tartrate-resistant acid phosphatase (TRAP) [20], and osteoclast-associated receptor (OSCAR) [21] are determined. The protein expression levels of eIF2α, ATF4, and nuclear factor of activated T cells cl (NFATcl) [22] are also determined NFATcl is a transcription factor, which is reportedly important for development and activation of osteoclasts in response to RANKL. RNA interference using siRNA specific to ATF4 and eIF2α is conducted to evaluate the role of ATF4 in osteoblastogenesis and eIF2α in osteoclastogenesis.

Without being bound by theory it is believed herein that the compounds described herein are efficacious, at least in part, due to the ability to inhibit the differentiation of RAW264.7 pre-osteoclasts to multi-nucleated osteoclasts. Without being bound by theory it is believed herein that the compounds described herein are efficacious, at least in part, due to the ability to block de-phosphorylation of eIF2α and elevate the level of p-eIF2α. It has been discovered herein that the growth arca, covered by multi-nucleated cells, is significantly reduced by salubrinal and guanabenz in a dose dependent manner. Partially silencing eIF2αusing RNA interference significantly suppressed salubrinal/guanabenz-driven reduction of NFATcl expression. Together with the stimulated development of MC3T3 E1 osteoblasts by an increase in ATF4 expression, the results herein support the conclusion that eIF2α mediated signaling plays a physiological role in osteoclastogenesis and osteoblastogenesis, and can be used in treating osteoporosis, fractures, and other bone diseases and disorders.

It is understood herein that both salubrinal and guanabenz interact with PP 1 and inhibit its activity of de-phosphorylating p-eTF2α. Further, guanabenz is reported to bind to PP1R15A subunit [14], while the exact binding site of salubrinal is not known. Guanabenz is also known as an α₂-adrenergic receptor agonist and used to treat hypertension [24]. Without being bound by theory, it is believed herein that the observed stimulation of osteoblastogenesis as well as attenuation of osteoclastogenesis by both agents strongly indicates that eIF2α-mediated signaling is central to regulation of ATF4 and NFATcl. This belief is also supported by the salubrinal-driven alterations in the mRNA levels of osteocalcin and TRAP, which are representative in development of osteoblasts and osteoclasts, respectively. Osteocalcin is synthesized solely by osteoblasts for matrix mineralization and calcium homeostasis [25], while TRAP is highly expressed in osteoclasts and its overexpression has been observed to cause bone loss in transgenic mice [26].

The elevation of p-eIF2α has been discovered herein to enhance the development of osteoblasts and mineralization of extracellular matrix. In response to various stresses such as oxidation, radiation, and stress to the endoplasmic reticulum, cells undergo either survival or apoptotic pathway [27]. As part of a pro-survival program, the level of p-eIF2α is raised followed by diminished translational efficiency to all proteins except for a limited group including ATF4 [5]. Salubrinal's action mimics the induction of pro-survival program without imposing damaging stresses, which result in the upregulation of ATF4 without inducing apoptosis. Since ATF4 is a transcription factor critical for osteoblastogenesis and bone formation, the effects of administration of salubrinal and guanabenz on the mRNA level of osteocalcin as well as the mineralization of extracellular matrix was examined. Silencing ATF4 using RNA interference significantly suppressed salubrinal-driven upregulation of osteocalcin expression. Thus, the result here is consistent with the previously reported role of salubrinal that stimulates new bone formation in the healing of bone wound [8].

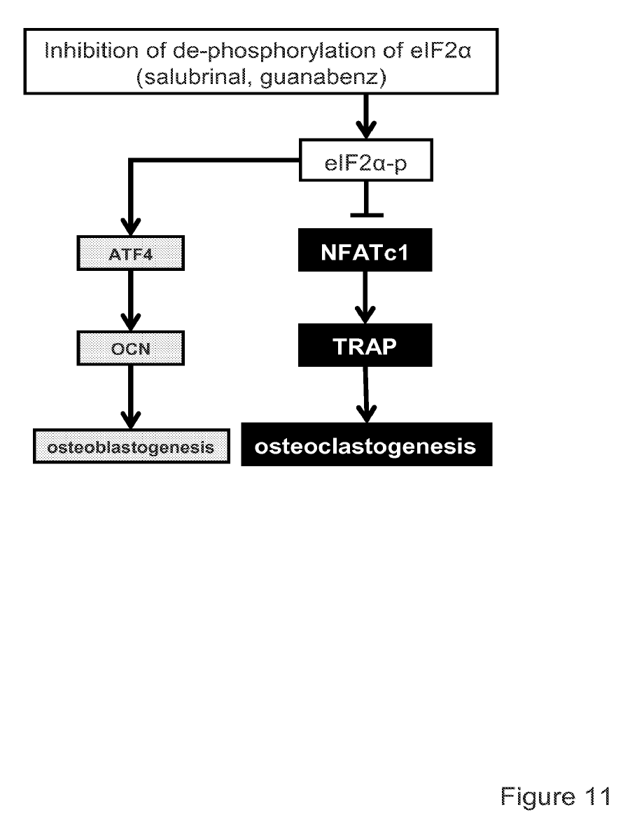

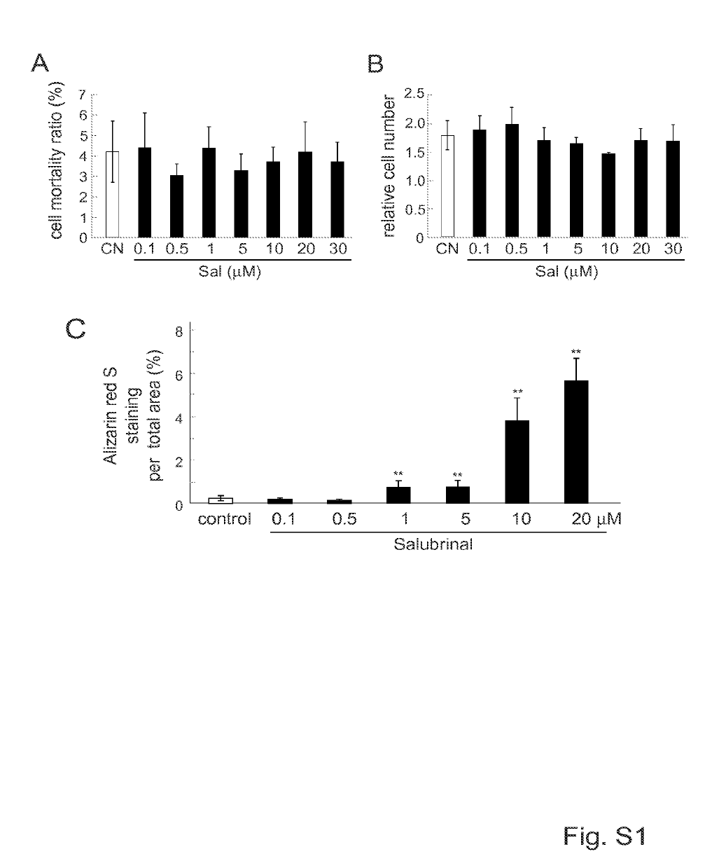

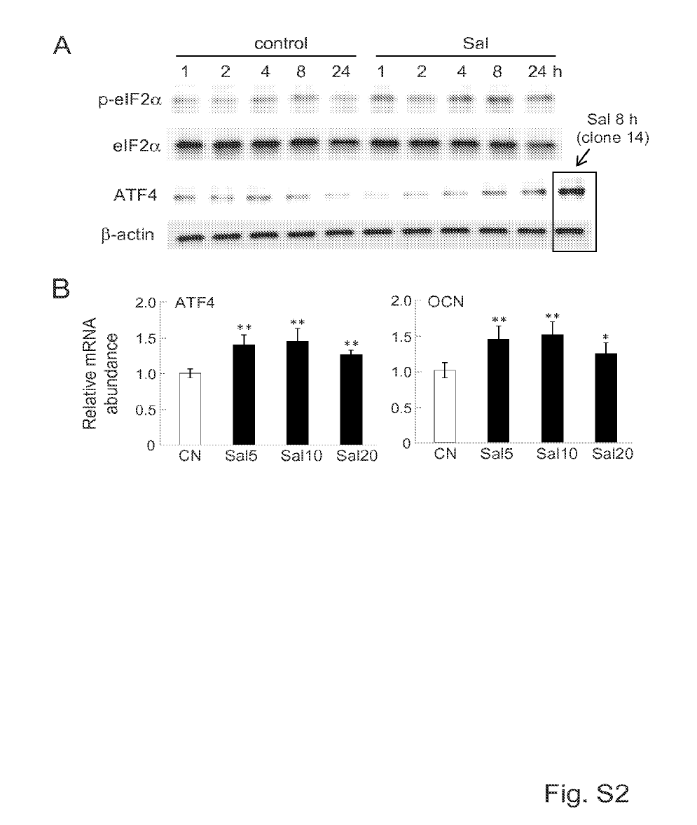

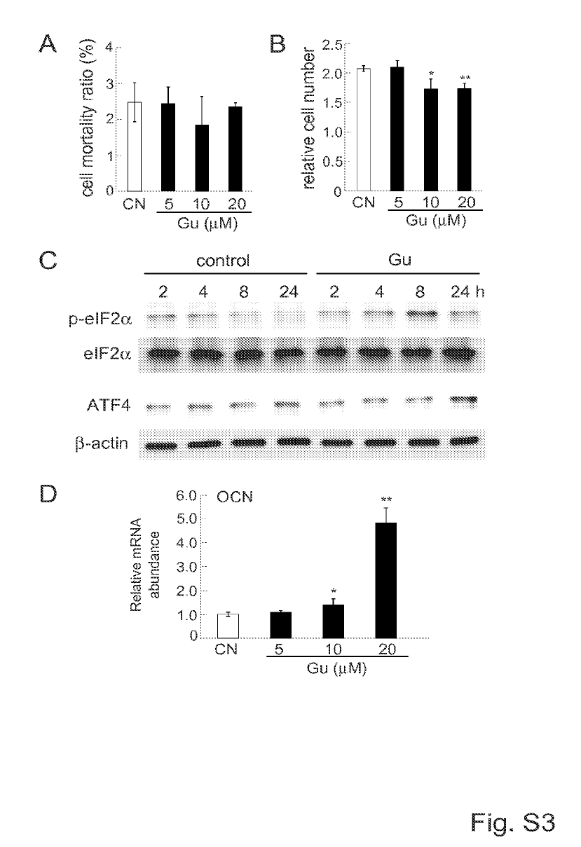

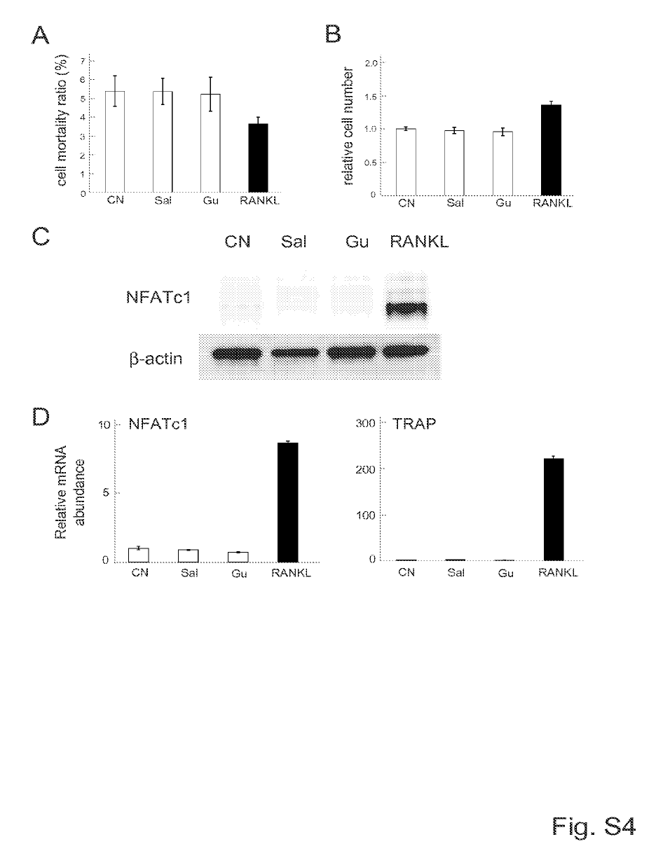

Without being bound by theory, a schematic diagram illustrating a proposed pathway of eIF2α-mediated signaling in osteoblastogenesis and osteoclastogenesis is presented (FIG. 11). Through inhibition of de-phosphorylation of eIF2α, salubrinal and guanabenz are capable of enhancing bone formation by activating ATF4, as well as reducing bone resorption by down-regulating NFATcl. Osteoclastogenesis is a multicomponent developmental process, in which active interactions take place between osteoblasts and osteoclasts. In the RANK/RANKL/OPG signaling pathway, for instance, osteoblasts provide RANKL that stimulates osteoclastogenesis. Since binding of RANKL to RANK is known to activate MAPKs and NFκB [28, 29], the potential role of ERK, p38, and NFκB in the eIF2α-mediated signaling. The levels of p-ERK, p-p38 MAPK, and p-NFκB together with p-eIF2α were evaluated by measuring the response to administration of 20 μM salubrinal. However, no detectable changes in the levels of their phosphorylated form were observed. Without being bound by theory, it is believed herein that salubrinal may activate transcription factors such as MafB (V-maf musculoaponeurotic fibrosarcoma oncogene homolog B), IRF8 (interferon regulatory factor 8), and Bcl6 (B-cell lymphoma 6), which arc known to be inhibitors of NFATcl [30-32]. Alternatively, microRNA and epigenetic changes such as histone modification regulate expression of NFATcl might be involved [33, 34]. For instance, H3K27 demethylase is reported to demethylate the site of H3K27me3 of NFATcl and stimulate RANKL-induced osteoclastogenesis [34].

Several illustrative embodiments of the invention are described by the following enumerated clauses:

1. A method for treating osteoporosis, fracture, or a bone defect in a host animal, the method comprising the step of administering to the host animal a therapeutically effective amount of one or more compounds capable of selectively inhibiting de-phosphorylation of eIF2α; where the amount is capable of (a) stimulating matrix deposition of osteoblasts, (b) upregulating activating transcription factor 4 (ATF4), (c) reducing expression of nuclear factor of activated T cells cl (NFATcl), (d) inhibiting differentiation of RAW264.7 cells to multi-nucleated osteoclasts, (e) stimulating osteoblastogenesis, or (f) inhibiting osteoclastogenesis, or a combination of the foregoing, or a pharmaceutically acceptable composition comprising the one or more compounds.

2. The method clause 1 wherein at least one compound is of the formula

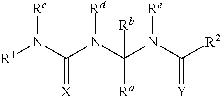

or a pharmaceutically acceptable salt thereof, wherein:

X is O or S;

Y is O or S;

R¹ is alkyl, cycloalkyl, alkenyl, cycloalkenyl, aryl, or heteroaryl, each of which is optionally substituted;

R² is alkyl, cycloalkyl, alkenyl, cycloalkenyl, aryl, heteroaryl, cycloalkylalkyl, cycloalkenylalkyl, arylalkyl, heteroarylalkyl, cycloalkylalkenyl, cycloalkenylalkenyl, arylalkenyl, or heteroarylalkenyl, each of which is optionally substituted;

R^{a} is optionally substituted alkyl;

R^{b} is H or optionally substituted C₁-C₆ alkyl;

R^{c}, R^{d}, and R^{e} are each independently selected from the group consisting of H, optionally substituted C₁-C₆ alkyl, acyl, or a prodrug capable of releasing the attached nitrogen in vivo.

3. The method clause 1 or 2 wherein at least one compound is of the formula

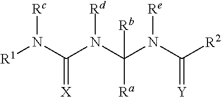

or a pharmaceutically acceptable salt thereof, wherein:

X is O or S;

Y is O or S;

R¹ is alkyl, cycloalkyl, alkenyl, cycloalkenyl, aryl, or heteroaryl, each of which is optionally substituted;

R² is alkyl, cycloalkyl, alkenyl, cycloalkenyl, aryl, heteroaryl, cycloalkylalkyl, cycloalkenylalkyl, arylalkyl, heteroarylalkyl, cycloalkylalkenyl, cycloalkenylalkenyl, arylalkenyl, or heteroarylalkenyl, each of which is optionally substituted;

R^{a} is optionally substituted alkyl;

R^{b} is H or optionally substituted C₁-C₆ alkyl;

R^{c}, R^{d}, and R^{e} are each independently selected from the group consisting of H, optionally substituted C₁-C₆ alkyl, acyl, or a prodrug capable of releasing the attached nitrogen in vivo;

or a composition thereof further comprising one or more carriers, diluents, or excipients, or a combination thereof.

4. The method of any one of the preceding clauses wherein at least one compound is of the formula

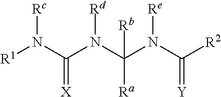

or a pharmaceutically acceptable salt thereof, wherein:

X is O or S;

Y is O or S;

R¹ is alkyl, cycloalkyl, alkenyl, cycloalkenyl, aryl, or heteroaryl, each of which is optionally substituted;

R² is alkyl, cycloalkyl, alkenyl, cycloalkenyl, aryl, heteroaryl, cycloalkylalkyl, cycloalkenylalkyl, arylalkyl, heteroarylalkyl, cycloalkylalkenyl, cycloalkenylalkenyl, arylalkenyl, or heteroarylalkenyl, each of which is optionally substituted;

R^{a} is optionally substituted alkyl;

R^{b} is H or optionally substituted C₁-C₆ alkyl;

R^{c}, R^{d}, and R^{e} are each independently selected from the group consisting of H, optionally substituted C₁-C₆ alkyl, acyl, or a prodrug capable of releasing the attached nitrogen in vivo.

5. The method of any one of the preceding clauses wherein R¹ is alkyl, aryl, or heteroaryl, each of which is optionally substituted.

6. The method of any one of the preceding clauses wherein R¹ is aryl or heteroaryl, each of which is optionally substituted.

7. The method of any one of the preceding clauses wherein R² is alkenyl, heteroaryl, cycloalkylalkyl, cycloalkenylalkyl, arylalkyl, heteroarylalkyl, cycloalkylalkenyl, cycloalkenylalkenyl, arylalkenyl, or heteroarylalkenyl, each of which is optionally substituted.

8. The method of any one of the preceding clauses wherein R² is alkenyl, cycloalkylalkyl, cycloalkenylalkyl, arylalkyl, heteroarylalkyl, cycloalkylalkenyl, cycloalkenylalkenyl, arylalkenyl, or heteroarylalkenyl, each of which is optionally substituted. 9. The method of any one of the preceding clauses wherein at least one compound is a compound of the formula

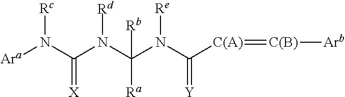

or a pharmaceutically acceptable salt thereof, wherein:

X and Y are independently O or S;

Ar^{a} and Ar^{b} are independently aryl or heteroaryl, each of which is optionally substituted;

R^{a} is optionally substituted alkyl;

R^{b} is H or optionally substituted C₁-C₆ alkyl;

R^{c}, R^{d}, and R^{e} are each independently selected from the group consisting of H, optionally substituted C₁-C₆ alkyl, acyl, and a prodrug capable of releasing the attached nitrogen in vivo to form the corresponding H or salt derivative thereof; and A and B are independently H, or optionally substituted C₁-C₆ alkyl 10. The method of clause 9 wherein the aryl is a bicyclic aryl. 11. The method of clause 9 or 10 wherein the heteroaryl is a bicyclic heteroaryl.

12. The method of any one of clauses 9 to 11 wherein the aryl or heteroaryl is optionally substituted with one or more substituents selected from the group consisting of halo, nitrile, or optionally substituted C₁-C₆ alkyl, C₁-C₆ alkoxy, C₁-C₆ haloalkyl, C₁-C₆ haloalkoxy, aryl, aryloxy, heterocyclyl, heterocyclyloxy, fused aryl, and fused heterocyclyl. 13. The method of any one of clauses 9 to 12 wherein the alkenyl has an E geometry.

14. The method of any one of the preceding clauses wherein at least one compound is of the formula

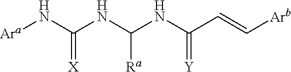

or a pharmaceutically acceptable salt thereof, wherein,

X is O or S;

Y is O or S;

Ar^{a} and Ar^{b} arc independently aryl or heteroaryl, each of which is optionally substituted; and

R^{a} is optionally substituted alkyl.

15. The method of clause 14 wherein the aryl is a bicyclic aryl.

16. The method of clause 14 or 15 wherein Ar^{a} is a bicyclic heteroaryl.

17. The method of any one of clauses 14 to 16 wherein the aryl or heteroaryl is optionally substituted with one or more substituents selected from the group consisting of halo, nitrile, or optionally substituted C₁-C₆ alkyl, C₁-C₆ alkoxy, C₁-C₆ haloalkyl, C₁-C₆ haloalkoxy, aryl, aryloxy, heterocyclyl, heterocyclyloxy, fused aryl, and fused heterocyclyl.

18. The method of any one of clauses 14 to 17 wherein R^{a} is haloalkyl.

19. The method of any one of clauses 14 to 18 wherein R^{a} is not trifluoromethyl.

20. The method of any one of clauses 14 to 19 wherein R^{a} is haloalkyl, where halo is selected from the group consisting of chloro and bromo, and combinations thereof.

21. The method of any one of clauses 14 to 20 wherein R^{c}, R^{d}, and R^{e} are each independently selected from the group consisting of H, optionally substituted C₁-C₆ alkyl, and acyl.

22. The method of any one of the preceding clauses wherein at least one compound is salubrinal, or an analog or a derivative thereof, or a pharmaceutically acceptable salt of the foregoing.

23. The method of any one of the preceding clauses wherein at least one compound is of the formula

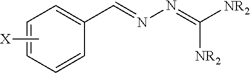

or a pharmaceutically acceptable salt thereof, wherein

X represents five substituents each independently selected from hydrogen, halo, hydroxy and derivatives thereof, amino and derivatives thereof, thio and derivatives thereof, carboxylate or a derivative thereof, sulfinyl or a derivative thereof, sulfonyl or a derivative thereof, phosphinyl or a derivative thereof, or phosphoryl or a derivative thereof, or alkyl, alkenyl, alkynyl, cycloalkyl, cycloalkenyl, heteroalkyl, heteroalkenyl, cycloheteroalkyl, cycloheteroalkenyl, aryl, heteroaryl, arylalkyl, or heteroarylalkyl, each of which is optionally substituted; and

R is independently selected in each instance from hydrogen, hydroxy and derivatives thereof, amino and derivatives thereof, carboxylate and derivatives thereof, sulfonyl and derivatives thereof, and alkyl, alkenyl, alkynyl, cycloalkyl, cycloalkenyl, heteroalkyl, heteroalkenyl, cycloheteroalkyl, cycloheteroalkenyl, aryl, heteroaryl, arylalkyl, and heteroarylalkyl, each of which is optionally substituted, and nitrogen prodrug forming groups.

24. The method of clause 23 wherein at least one X is halo, such as chloro.

25. The method of clause 23 wherein at least one X is optionally substituted alkyl, such as methyl or trifluoromethyl.

26. The method of clause 23 wherein at least one X is optionally substituted alkoxy, such as methoxy or trifluoromethoxy.

27. The method of any one of the preceding clauses wherein at least one compound is of the formula

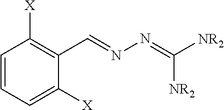

or a pharmaceutically acceptable salt thereof

28. The method of clause 27 wherein each X is halo.

29. The method of clause 27 wherein each X is chloro.

30. The method of any one of clauses 27 to 29 wherein each R is hydrogen

31. The method of any one of clauses 27 to 30 wherein at least one compound is guanabenz or a pharmaceutically acceptable salt thereof.

32. The method of any one of the preceding clauses wherein the composition further comprises one or more carriers, diluents, or excipients, or a combination thereof.

33. The method of any one of the preceding clauses wherein the host animal is a human.

The compounds described herein may contain one or more chiral centers, or may otherwise be capable of existing as multiple stereoisomers. It is to be understood that in one embodiment, the invention described herein is not limited to any particular sterochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

As used herein, the term “alkyl” includes a chain of carbon atoms, which is optionally branched. As used herein, the term “alkenyl” and “alkynyl” includes a chain of carbon atoms, which is optionally branched, and includes at least one double bond or triple bond, respectively. It is to be understood that alkynyl may also include one or more double bonds. It is to be further understood that in certain embodiments, alkyl is advantageously of limited length, including C₁-C₂₄, C₁-C₁₂, C₁-C₈, C₁-C₆, and C₁-C₄. Illustratively, such particularly limited length alkyl groups, including C₁-C₈, C₁-C₆, and C₁-C₄ may be referred to as lower alkyl. It is to be further understood that in certain embodiments alkenyl and/or alkynyl may each be advantageously of limited length, including C₂-C₂₄, C₂-C₁₂, C₂-C₈, C₂-C₆, and C₂-C₄. Illustratively, such particularly limited length alkenyl and/or alkynyl groups, including C₂-C₈, C₂-C₆, and C₂-C₄ may be referred to as lower alkenyl and/or alkynyl. It is appreciated herein that shorter alkyl, alkenyl, and/or alkynyl groups may add less lipophilicity to the compound and accordingly will have different pharmacokinetic behavior. In embodiments of the invention described herein, it is to be understood, in each case, that the recitation of alkyl refers to alkyl as defined herein, and optionally lower alkyl. In embodiments of the invention described herein, it is to be understood, in each case, that the recitation of alkenyl refers to alkenyl as defined herein, and optionally lower alkenyl. In embodiments of the invention described herein, it is to be understood, in each case, that the recitation of alkynyl refers to alkynyl as defined herein, and optionally lower alkynyl. Illustrative alkyl, alkenyl, and alkynyl groups are, but not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like, and the corresponding groups containing one or more double and/or triple bonds, or a combination thereof.

As used herein, the term “alkylene” includes a divalent chain of carbon atoms, which is optionally branched. As used herein, the term “alkenylene” and “alkynylene” includes a divalent chain of carbon atoms, which is optionally branched, and includes at least one double bond or triple bond, respectively. It is to be understood that alkynylene may also include one or more double bonds. It is to be further understood that in certain embodiments, alkylene is advantageously of limited length, including C₁-C₂₄, C₁-C₁₂, C₁-C₈, C₁-C₆, and C₁-C₄. Illustratively, such particularly limited length alkylene groups, including C₁-C₈, C₁-C₆, and C₁-C₄ may be referred to as lower alkylene. It is to be further understood that in certain embodiments alkenylene and/or alkynylene may each be advantageously of limited length, including C₂⁻C₂₄, C₂⁻C₁₂, C₂-C₈, C₂-C₆, and C₂-C₄. Illustratively, such particularly limited length alkenylene and/or alkynylene groups, including C₂-C₈, C₂-C₆, and C₂-C₄ may be referred to as lower alkenylene and/or alkynylene. It is appreciated herein that shorter alkylene, alkenylene, and/or alkynylene groups may add less lipophilicity to the compound and accordingly will have different pharmacokinetic behavior. In embodiments of the invention described herein, it is to be understood, in each case, that the recitation of alkylene, alkenylene, and alkynylene refers to alkylene, alkenylene, and alkynylene as defined herein, and optionally lower alkylene, alkenylene, and alkynylene. Illustrative alkyl groups are, but not limited to, methylene, ethylene, n-propylene, isopropylene, n-butylene, isobutylene, sec-butylene, pentylene, 1,2-pentylene, 1,3-pentylene, hexylene, heptylene, octylene, and the like.

As used herein, the term “linker” includes is a chain of atoms that connects two or more functional parts of a molecule to form a conjugate. Illustratively, the chain of atoms is selected from C, N, O, S, Si, and P, or C, N, O, S, and P, C, N, O, and S. The chain of atoms covalently connects different functional capabilities of the conjugate, such as binding ligands, drugs, diagnostic agents, imaging agents, and the like. The linker may have a wide variety of lengths, such as in the range from about 2 to about 100 atoms in the contiguous backbone. The atoms used in forming the linker may be combined in all chemically relevant ways, such as chains of carbon atoms forming alkylene, alkenylene, and alkynylene groups, and the like; chains of carbon and oxygen atoms forming ethers, polyoxyalkylene groups, or when combined with carbonyl groups forming esters and carbonates, and the like; chains of carbon and nitrogen atoms forming amines, imines, polyamines, hydrazines, hydrazones, or when combined with carbonyl groups forming amides, ureas, semicarbazides, carbazides, and the like; chains of carbon, nitrogen, and oxygen atoms forming alkoxyamines, alkoxylamines, or when combined with carbonyl groups forming urethanes, amino acids, acyloxylamines, hydroxamic acids, and the like; and many others. In addition, it is to be understood that the atoms forming the chain in each of the foregoing illustrative embodiments may be either saturated or unsaturated, thus forming single, double, or triple bonds, such that for example, alkanes, alkenes, alkynes, imines, and the like may be radicals that are included in the linker. In addition, it is to be understood that the atoms forming the linker may also be cyclized upon each other or be part of cyclic structure to form divalent cyclic structures that form the linker, including cyclo alkanes, cyclic ethers, cyclic amines, and other heterocycles, arylcncs, heteroarylcnes, and the like in the linker. In this latter arrangement, it is to be understood that the linker length may be defined by any pathway through the one or more cyclic structures. Illustratively, the linker length is defined by the shortest pathway through the each one of the cyclic structures. It is to be understood that the linkers may be optionally substituted at any one or more of the open valences along the chain of atoms, such as optional substituents on any of the carbon, nitrogen, silicon, or phosphorus atoms. It is also to be understood that the linker may connect the two or more functional parts of a molecule to form a conjugate at any open valence, and it is not necessary that any of the two or more functional parts of a molecule forming the conjugate are attached at any apparent end of the linker.

As used herein, the term “cycloalkyl” includes a chain of carbon atoms, which is optionally branched, where at least a portion of the chain in cyclic. It is to be understood that cycloalkylalkyl is a subset of cycloalkyl. It is to be understood that cycloalkyl may be polycyclic. Illustrative cycloalkyl include, but are not limited to, cyclopropyl, cyclopentyl, cyclohexyl, 2-methylcyclopropyl, cyclopentyleth-2-yl, adamantyl, and the like. As used herein, the term “cycloalkenyl” includes a chain of carbon atoms, which is optionally branched, and includes at least one double bond, where at least a portion of the chain in cyclic. It is to be understood that the one or more double bonds may be in the cyclic portion of cycloalkenyl and/or the non-cyclic portion of cycloalkenyl. It is to be understood that cycloalkenylalkyl and cycloalkylalkenyl are each subsets of cycloalkenyl. It is to be understood that cycloalkyl may be polycyclic. Illustrative cycloalkenyl include, but are not limited to, cyclopentenyl, cyclohexylethen-2-yl, cycloheptenylpropenyl, and the like. It is to be further understood that chain forming cycloalkyl and/or cycloalkenyl is advantageously of limited length, including C₃-C₂₄, C₃-C₁₂, C₃-C₈, C₃-C₆, and C₅-C₆. It is appreciated herein that shorter alkyl and/or alkenyl chains forming cycloalkyl and/or cycloalkenyl, respectively, may add less lipophilicity to the compound and accordingly will have different pharmacokinetic behavior.

As used herein, the term “heteroalkyl” includes a chain of atoms that includes both carbon and at least one heteroatom, and is optionally branched. Illustrative heteroatoms include nitrogen, oxygen, and sulfur. In certain variations, illustrative heteroatoms also include phosphorus, and selenium. As used herein, the term “cycloheteroalkyl” including heterocyclyl and heterocycle, includes a chain of atoms that includes both carbon and at least one heteroatom, such as heteroalkyl, and is optionally branched, where at least a portion of the chain is cyclic. Illustrative heteroatoms include nitrogen, oxygen, and sulfur. In certain variations, illustrative heteroatoms also include phosphorus, and selenium. Illustrative cycloheteroalkyl include, but are not limited to, tetrahydrofuryl, pyrrolidinyl, tetrahydropyranyl, piperidinyl, morpholinyl, piperazinyl, homopiperazinyl, quinuclidinyl, and the like.

As used herein, the term “aryl” includes monocyclic and polycyclic aromatic carbocyclic groups, each of which may be optionally substituted. Illustrative aromatic carbocyclic groups described herein include, but are not limited to, phenyl, naphthyl, and the like. As used herein, the term “heteroaryl” includes aromatic heterocyclic groups, each of which may be optionally substituted. Illustrative aromatic heterocyclic groups include, but are not limited to, pyridinyl, pyrimidinyl, pyrazinyl, triazinyl, tetrazinyl, quinolinyl, quinazolinyl, quinoxalinyl, thienyl, pyrazolyl, imidazolyl, oxazolyl, thiazolyl, isoxazolyl, isothiazolyl, oxadiazolyl, thiadiazolyl, triazolyl, benzimidazolyl, benzoxazolyl, benzthiazolyl, benzisoxazolyl, benzisothiazolyl, and the like.

As used herein, the term “amino” includes the group NH₂, alkylamino, and dialkylamino, where the two alkyl groups in dialkylamino may be the same or different, i.e. alkylalkylamino Illustratively, amino includes methylamino, ethylamino, dimethylamino, methylethylamino, and the like. In addition, it is to be understood that when amino modifies or is modified by another term, such as aminoalkyl, or acylamino, the above variations of the term amino are included therein. Illustratively, aminoalkyl includes H₂N-alkyl, methylaminoalkyl, ethylaminoalkyl, dimethylaminoalkyl, methylethylaminoalkyl, and the like. Illustratively, acylamino includes acylmethylamino, acylethylamino, and the like.

As used herein, the term “amino and derivatives thereof” includes amino as described herein, and alkylamino, alkenylamino, alkynylamino, heteroalkylamino, heteroalkenylamino, heteroalkynylamino, cycloalkylamino, cycloalkenylamino, cycloheteroalkylamino, cycloheteroalkenylamino, arylamino, arylalkylamino, arylalkenylamino, arylalkynylamino, heteroarylamino, heteroarylalkylamino, heteroarylalkenylamino, heteroarylalkynylamino, acylamino, and the like, each of which is optionally substituted. The term “amino derivative” also includes urea, carbamatc, and the like.

As used herein, the term “hydroxy and derivatives thereof” includes OH, and alkyloxy, alkenyloxy, alkynyloxy, heteroalkyloxy, heteroalkenyloxy, heteroalkynyloxy, cycloalkyloxy, cycloalkenyloxy, cycloheteroalkyloxy, cycloheteroalkenyloxy, aryloxy, arylalkyloxy, arylalkenyloxy, arylalkynyloxy, heteroaryloxy, heteroarylalkyloxy, heteroarylalkenyloxy, heteroarylalkynyloxy, acyloxy, and the like, each of which is optionally substituted. The term “hydroxy derivative” also includes carbamate, and the like.

As used herein, the term “thio and derivatives thereof” includes SH, and alkylthio, alkenylthio, alkynylthio, heteroalkylthio, heteroalkenylthio, heteroalkynylthio, cycloalkylthio, cycloalkenylthio, cycloheteroalkylthio, cycloheteroalkenylthio, arylthio, arylalkylthio, arylalkenylthio, arylalkynylthio, heteroarylthio, heteroarylalkylthio, heteroarylalkenylthio, heteroarylalkynylthio, acylthio, and the like, each of which is optionally substituted. The term “thio derivative” also includes thiocarbamate, and the like.

As used herein, the term “acyl” includes formyl, and alkylcarbonyl, alkenylcarbonyl, alkynylcarbonyl, heteroalkylcarbonyl, heteroalkenylcarbonyl, heteroalkynylcarbonyl, cycloalkylcarbonyl, cycloalkenylcarbonyl, cycloheteroalkylcarbonyl, cycloheteroalkenylcarbonyl, arylcarbonyl, arylalkylcarbonyl, arylalkenylcarbonyl, arylalkynylcarbonyl, heteroarylcarbonyl, heteroarylalkylcarbonyl, heteroarylalkenylcarbonyl, heteroarylalkynylcarbonyl, acylcarbonyl, and the like, each of which is optionally substituted.

As used herein, the term “carbonyl and derivatives thereof” includes the group C(O), C(S), C(NH) and substituted amino derivatives thereof.

As used herein, the term “carboxylic acid and derivatives thereof” includes the group CO₂H and salts thereof, and esters and amides thereof, and CN.

As used herein, the term “sulfinic acid or a derivative thereof” includes SO₂H and salts thereof, and esters and amides thereof.

As used herein, the term “sulfonic acid or a derivative thereof” includes SO₃H and salts thereof, and esters and amides thereof.

As used herein, the term “sulfonyl” includes alkylsulfonyl, alkenylsulfonyl, alkynylsulfonyl, heteroalkylsulfonyl, heteroalkenylsulfonyl, heteroalkynylsulfonyl, cycloalkylsulfonyl, cycloalkenylsulfonyl, cycloheteroalkylsulfonyl, cycloheteroalkenylsulfonyl, arylsulfonyl, arylalkylsulfonyl, arylalkenylsulfonyl, arylalkynylsulfonyl, heteroarylsulfonyl, heteroarylalkylsulfonyl, heteroarylalkenylsulfonyl, heteroarylalkynylsulfonyl, acylsulfonyl, and the like, each of which is optionally substituted.

As used herein, the term “phosphinic acid or a derivative thereof” includes P(R)O₂H and salts thereof, and esters and amides thereof, where R is alkyl, alkenyl, alkynyl, cycloalkyl, cycloalkcnyl, heteroalkyl, heteroalkcnyl, cycloheteroalkyl, cyclohcteroalkcnyl, aryl, heteroaryl, arylalkyl, or heteroarylalkyl, each of which is optionally substituted.

As used herein, the term “phosphonic acid or a derivative thereof” includes PO₃H₂ and salts thereof, and esters and amides thereof.

As used herein, the term “hydroxylamino and derivatives thereof” includes NHOH, and alkyloxylNH alkenyloxylNH alkynyloxylNH heteroalkyloxylNH heteroalkenyloxylNH heteroalkynyloxylNH cycloalkyloxylNH cycloalkenyloxylNH cycloheteroalkyloxylNH cycloheteroalkenyloxylNH aryloxylNH arylalkyloxylNH arylalkenyloxylNH arylalkynyloxylNH heteroaryloxylNH heteroarylalkyloxylNH heteroarylalkenyloxylNH heteroarylalkynyloxylNH acyloxy, and the like, each of which is optionally substituted.

As used herein, the term “hydrazino and derivatives thereof” includes alkylNHNH, alkenylNHNH, alkynylNHNH, heteroalkylNHNH, heteroalkenylNHNH, heteroalkynylNHNH, cycloalkylNHNH, cycloalkenylNHNH, cycloheteroalkylNHNH, cycloheteroalkenylNHNH, arylNHNH, arylalkylNHNH, arylalkenylNHNH, arylalkynylNHNH, heteroarylNHNH, heteroaiylalkylNHNH, heteroarylalkenylNHNH, heteroaiylalkynylNHNH, acylNHNH, and the like, each of which is optionally substituted.

The term “optionally substituted” as used herein includes the replacement of hydrogen atoms with other functional groups on the radical that is optionally substituted. Such other functional groups illustratively include, but are not limited to, amino, hydroxyl, halo, thiol, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

As used herein, the terms “optionally substituted aryl” and “optionally substituted heteroaryl” include the replacement of hydrogen atoms with other functional groups on the aryl or heteroaryl that is optionally substituted. Such other functional groups illustratively include, but are not limited to, amino, hydroxy, halo, thio, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxy, thio, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

Illustrative substituents include, but are not limited to, a radical —(CH₂)ₓZ^{X}, where x is an integer from 0-6 and Z^{X} is selected from halogen, hydroxy, alkanoyloxy, including C₁-C₆ alkanoyloxy, optionally substituted aroyloxy, alkyl, including C₁-C₆ alkyl, alkoxy, including C₁-C₆ alkoxy, cycloalkyl, including C₃-C₈ cycloalkyl, cycloalkoxy, including C₃-C₈ cycloalkoxy, alkenyl, including C₂-C₆ alkenyl, alkynyl, including C₂-C₆ alkynyl, haloalkyl, including C₁-C₆ haloalkyl, haloalkoxy, including C₁-C₆ haloalkoxy, halocycloalkyl, including C₃-C₈ halocycloalkyl, halocycloalkoxy, including C₃-C₈ halocycloalkoxy, amino, C₁-C₆ alkylamino, (C₁-C₆ alkyl)(C₁-C₆ alkyl)amino, alkylcarbonylamino, N—(C₁-C₆ alkyl)alkylcarbonylamino, aminoalkyl, C₁-C₆ alkylaminoalkyl, (C₁-C₆ alkyl)(C₁-C₆ alkyl)aminoalkyl, alkylcarbonylaminoalkyl, N—(C₁-C₆ alkyl)alkylcarbonylaminoalkyl, cyano, and nitro; or Z^{X} is selected from —CO₂R⁴ and —CONR⁵R⁶, where R⁴, R⁵, and R⁶ are each independently selected in each occurrence from hydrogen, C₁-C₆ alkyl, aryl-C₁-C₆ alkyl, and heteroaryl-C₁-C₆ alkyl.

The term “prodrug” as used herein generally refers to any compound that when administered to a biological system generates a biologically active compound as a result of one or more spontaneous chemical reaction(s), enzyme-catalyzed chemical reaction(s), and/or metabolic chemical reaction(s), or a combination thereof. In vivo, the prodrug is typically acted upon by an enzyme (such as esterases, amidases, phosphatases, and the like), simple biological chemistry, or other process in vivo to liberate or regenerate the more pharmacologically active drug. This activation may occur through the action of an endogenous host enzyme or a non-endogenous enzyme that is administered to the host preceding, following, or during administration of the prodrug. Additional details of prodrug use are described in U.S. Pat. No. 5,627,165; and Pathalk et al., Enzymic protecting group techniques in organic synthesis, Stereosel. Biocatal. 775-797 (2000). It is appreciated that the prodrug is advantageously converted to the original drug as soon as the goal, such as targeted delivery, safety, stability, and the like is achieved, followed by the subsequent rapid elimination of the released remains of the group forming the prodrug.

Prodrugs may be prepared from the compounds described herein by attaching groups that ultimately cleave in vivo to one or more functional groups present on the compound, such as —OH—, —SH, —CO₂H, —NR₂. Illustrative prodrugs include but are not limited to carboxylate esters where the group is alkyl, aryl, arylalkyl, heteroaryl, heteroarylalkyl, acyloxyalkyl, alkoxycarbonyloxyalkyl as well as esters of hydroxyl, thiol and amines where the group attached is an acyl group, an alkoxycarbonyl, aminocarbonyl, phosphate or sulfate. Illustrative esters, also referred to as active esters, include but are not limited to 1-indanyl, N-oxysuccinimide; acyloxyalkyl groups such as acetoxymethyl, pivaloyloxymethyl, β-acetoxyethyl, β-pivaloyloxyethyl, 1-(cyclohexylearbonyloxy)prop-1-yl, (1-aminoethyl)carbonyloxymethyl, and the like; alkoxycarbonyloxyalkyl groups, such as ethoxycarbonyloxymethyl, α-ethoxycarbonyloxyethyl, β-ethoxycarbonyloxycthyl, and the like; dialkylaminoalkyl groups, including di-lower alkylamino alkyl groups, such as dimethylaminomethyl, dimethylaminoethyl, diethylaminomethyl, diethylaminoethyl, and the like; 2-(alkoxycarbonyl)-2-alkenyl groups such as 2-(isobutoxycarbonyl) pent-2-enyl, 2-(ethoxycarbonyl)but-2-enyl, and the like; and lactone groups such as phthalidyl, dimethoxyphthalidyl, and the like.

Further illustrative prodrugs contain a chemical moiety, such as an amide or phosphorus group functioning to increase solubility and/or stability of the compounds described herein. Further illustrative prodrugs for amino groups include, but are not limited to, (C₃-C₂₀)alkanoyl; halo-(C₃-C₂₀)alkanoyl; (C₃-C₂₀)alkenoyl; (C₄-C₇)cycloalkanoyl; (C₃-C₆-cycloalkyl(C₂-C₁₆)alkanoyl; optionally substituted aroyl, such as unsubstituted aroyl or aroyl substituted by 1 to 3 substituents selected from the group consisting of halogen, cyano, trifluoromethanesulphonyloxy, (C₁-C₃)alkyl and (C₁-C₃)alkoxy, each of which is optionally further substituted with one or more of 1 to 3 halogen atoms; optionally substituted aryl(C₂-C₁₆)alkanoyl and optionally substituted heteroaryl(C₂-C₁₆)alkanoyl, such as the aryl or heteroaryl radical being unsubstituted or substituted by 1 to 3 substituents selected from the group consisting of halogen, (C₁-C₃)alkyl and (C₁-C₃)alkoxy, each of which is optionally further substituted with 1 to 3 halogen atoms; and optionally substituted heteroarylalkanoyl having one to three heteroatoms selected from O, S and N in the heteroaryl moiety and 2 to 10 carbon atoms in the alkanoyl moiety, such as the heteroaryl radical being unsubstituted or substituted by 1 to 3 substituents selected from the group consisting of halogen, cyano, trifluoromethanesulphonyloxy, (C₁-C₃)alkyl, and (C₁-C₃)alkoxy, each of which is optionally further substituted with 1 to 3 halogen atoms. The groups illustrated are exemplary, not exhaustive, and may be prepared by conventional processes.

It is understood that the prodrugs themselves may not possess significant biological activity, but instead undergo one or more spontaneous chemical reaction(s), enzyme-catalyzed chemical reaction(s), and/or metabolic chemical reaction(s), or a combination thereof after administration in vivo to produce the compound described herein that is biologically active or is a precursor of the biologically active compound. However, it is appreciated that in some cases, the prodrug is biologically active. It is also appreciated that prodrugs may often serves to improve drug efficacy or safety through improved oral bioavailability, pharmacodynamic half-life, and the like. Prodrugs also refer to derivatives of the compounds described herein that include groups that simply mask undesirable drug properties or improve drug delivery. For example, one or more compounds described herein may exhibit an undesirable property that is advantageously blocked or minimized may become pharmacological, pharmaceutical, or pharmacokinetic barriers in clinical drug application, such as low oral drug absorption, lack of site specificity, chemical instability, toxicity, and poor patient acceptance (bad taste, odor, pain at injection site, and the like), and others. It is appreciated herein that a prodrug, or other strategy using reversible derivatives, can be useful in the optimization of the clinical application of a drug.

As used herein, the term “composition” generally refers to any product comprising the specified ingredients in the specified amounts, as well as any product which results, directly or indirectly, from combinations of the specified ingredients in the specified amounts. It is to be understood that the compositions described herein may be prepared from isolated compounds described herein or from salts, solutions, hydrates, solvates, and other forms of the compounds described herein. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. Accordingly, such pharmaceutical compositions that recite compounds described herein are to be understood to include each of, or any combination of, the various morphological forms and/or solvate or hydrate forms of the compounds described herein. Illustratively, compositions may include one or more carriers, diluents, and/or excipients. The compounds described herein, or compositions containing them, may be formulated in a therapeutically effective amount in any conventional dosage forms appropriate for the methods described herein. The compounds described herein, or compositions containing them, including such formulations, may be administered by a wide variety of conventional routes for the methods described herein, and in a wide variety of dosage formats, utilizing known procedures (see generally, Remington: The Science and Practice of Pharmacy, (21^{st} ed., 2005)).

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

It is also appreciated that the therapeutically effective amount, whether referring to monotherapy or combination therapy, is advantageously selected with reference to any toxicity, or other undesirable side effect, that might occur during administration of one or more of the compounds described herein. Further, it is appreciated that the co-therapies described herein may allow for the administration of lower doses of compounds that show such toxicity, or other undesirable side effect, where those lower doses arc below thresholds of toxicity or lower in the therapeutic window than would otherwise be administered in the absence of a cotherapy. In addition to the illustrative dosages and dosing protocols described herein, it is to be understood that an effective amount of any one or a mixture of the compounds described herein can be readily determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “administering” as used herein includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically-acceptable carriers, adjuvants, and/or vehicles.

Illustrative formats for oral administration include tablets, capsules, elixirs, syrups, and the like.

Illustrative routes for parenteral administration include intravenous, intraarterial, intraperitoneal, epidurial, intraurethral, intrasternal, intramuscular and subcutaneous, as well as any other art recognized route of parenteral administration.

Illustratively, administering includes local use, such as when administered locally to the site of disease, injury, or defect, or to a particular organ or tissue system. Illustrative local administration may be performed during open surgery, or other procedures when the site of disease, injury, or defect is accessible. Alternatively, local administration may be performed using parenteral delivery where the compound or compositions described herein are deposited locally to the site without general distribution to multiple other non-target sites in the patient being treated. It is further appreciated that local administration may be directly in the injury site, or locally in the surrounding tissue. Similar variations regarding local delivery to particular tissue types, such as organs, and the like, are also described herein. Illustratively, compounds may be administered directly to the nervous system including, but not limited to, intracerebral, intraventricular, intracerebroventricular, intrathecal, intracisternal, intraspinal and/or peri-spinal routes of administration by delivery via intracranial or intravertebral needles and/or catheters with or without pump devices.

Depending upon the disease as described herein, the route of administration and/or whether the compounds and/or compositions are administered locally or systemically, a wide range of permissible dosages are contemplated herein, including doses falling in the range from about 1 μg/kg to about 1 g/kg. The dosages may be single or divided, and may administered according to a wide variety of protocols, including q.d., b.i.d., t.i.d., or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In making the pharmaceutical compositions of the compounds described herein, a therapeutically effective amount of one or more compounds in any of the various forms described herein may be mixed with one or more excipients, diluted by one or more excipients, or enclosed within such a carrier which can be in the form of a capsule, sachet, paper, or other container. Excipients may serve as a diluent, and can be solid, semi-solid, or liquid materials, which act as a vehicle, carrier or medium for the active ingredient. Thus, the formulation compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders. The compositions may contain anywhere from about 0.1% to about 99.9% active ingredients, depending upon the selected dose and dosage form.

The following examples further illustrate specific embodiments of the invention; however, the following illustrative examples should not be interpreted in any way to limit the invention.

### EXAMPLES

METHOD EXAMPLE. Cell culture. MC3T3 E1 mouse osteoblast-like cells (clone 14—MC3T3 E1-14; and no clonal cells in supplementary figures), and RAW264.7 mouse pre-osteoclast (monocyte/macrophage) cells were cultured in αMEM containing 10% fetal bovine serum and antibiotics (50 units/ml penicillin, and 50 μg/ml streptomycin; Life Technologies, Grand Island, N.Y., USA). Cells were maintained at 37° C. and 5% CO₂ in a humidified incubator. Cell mortality and live cell numbers were determined 24 h after the treatment with 20 ng/ml RANKL (PeproTech, Rocky Hills, N.C., USA) in response to 0.1-20 μM salubrinal or 1-20 μM guanabenz acetate (Tocris Bioscience, Ellisville, Mo., USA). Cells were stained with trypan blue and the numbers of live and dead cells were counted using a hemacytometer.

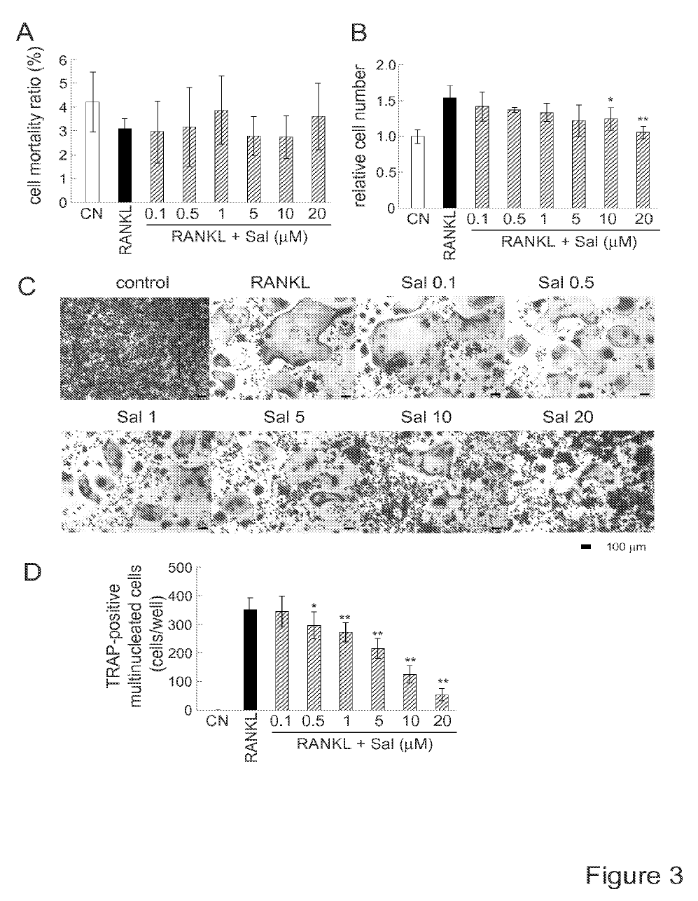

METHOD EXAMPLE. Inhibition of osteoclastogenesis of RAW264.7 cells by salubrinal. The primary aim of this study is to evaluate the effects of salubrinal on osteoclastogenesis. In response to 0.1-20 μM salubrinal for 24 h, cell mortality and live cell numbers of RAW264.7 pre-osteoclasts was examined. Cell mortality ratio did not present statistically significant differences in the presence and absence of RANKL (FIG. 3A). The number of live cells was increased by ˜50% by incubation with RANKL, and administration of 10-20 μM salubrinal reduced the numbers approximately by 10% (FIG. 3B). Consistent with the stimulatory role of RANKL, the number of TRAP-positive multi-nucleated cells was substantially increased by addition of RANKL. However, administration of 0.5 μM to 20 μM salubrinal reduced the number of TRAP-positive cells in a dose dependent manner (FIG. 3C & 3D).

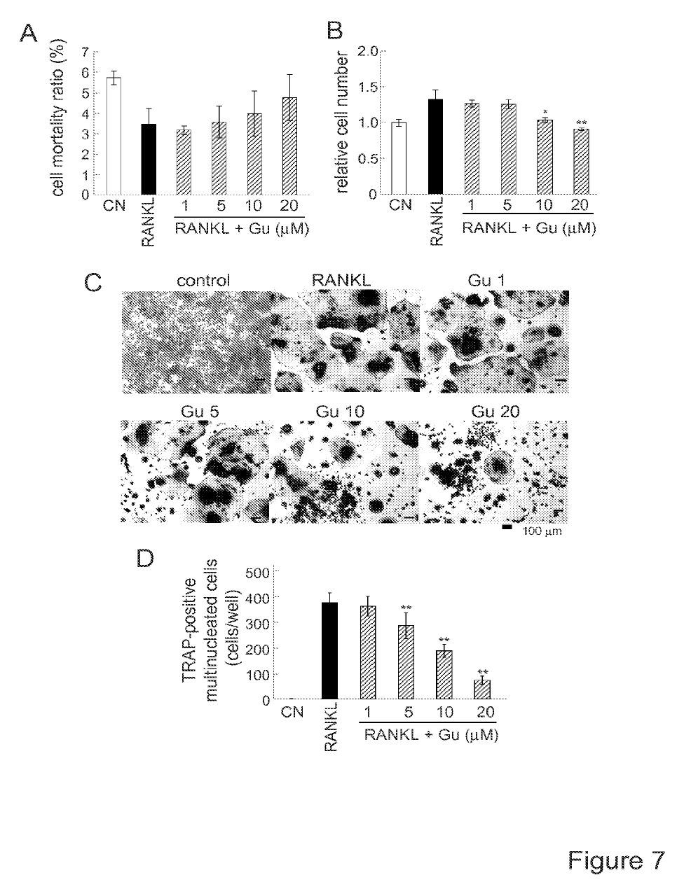

METHOD EXAMPLE. Inhibitory effects of guanabenz on osteoclastogenesis of RAW264.7 cells. To further examine a potential involvement of p-eIF2α in regulation of osteoclastogenesis, we employed guanabenz that also acts as an inhibitor of de-phosphorylation of eIF2α was evaluated. Administration of 1 and 5 μM guanabenz did not alter cell mortality and the number of live cells, although its administration at 10 and 20 μM reduced the number of live cells in 24 h (FIG. 7A & 7B). Consistent with salubrinal's inhibitory action, guanabenz also attenuated osteoclastogenesis of RAW264.7 cells in a dose dependent manner (FIG. 7C & 7D). Compared to the number of TRAP-positive multi-nucleated cells of 377 39 (RANKL only), guanabenz reduced the number of differentiated osteoclasts to 364±38 (1 μM), 288±51 (5 μM), 189±25 (10 μM), and 73±16 (20 μM).

METHOD EXAMPLE. Quantitative real-time PCR. Total RNA was extracted using an RNeasy Plus mini kit (Qiagen, Germantown, Md., USA). Reverse transcription was conducted with high capacity cDNA reverse transcription kits (Applied Biosystems, Carlsbad, Calif., USA), and quantitative real-time PCR was performed using ABI 7500 with Power SYBR green PCR master mix kits (Applied Biosystems). We evaluated mRNA levels of ATF4, Osteocalcin (OCN), NFATcl, c-Fos, tartrate-resistant acid phosphatase (TRAP), and osteoclast-associated receptor (OSCAR) with the PCR primers listed in Table 1. GAPDH was used for internal control. The relative mRNA abundance for the selected genes with respect to the level of GAPDH mRNA was expressed as a ratio of S_{treated}/S_{control}, where S_{treated}=mRNA level for the cells treated with chemical agents, and S_{control}=mRNA level for control cells [23].

TABLE 1
Real-time PCR primers used in this study.

| target | forward primer | backward primer |
|---|---|---|
| ATF4 | 5′-TGGCGAGTGTA | 5′-TCTTCCCCCTTG |
| | AGGAGCTAGAAA-3′ | CCTTACG-3′ |
| OCN | 5′-CCGGGAGCAGT | 5′-AGGCGGTCTTCA |
| | GTGAGCTTA-3′ | AGCCATACT-3′ |
| NFATc1 | 5′-GGTGCTGTCTG | 5′-GCGGAAAGGTGG |
| | GCCATAACT-3′ | TATCTCAA-3′ |
| c-Fos | 5′-AGGCCCAGTGG | 5′-CCAGTCTGCTGC |
| | CTCAGAGA-3′ | ATAGAAGGAA-3′ |
| TRAP | 5′-TCCTGGCTCAA | 5′-ACATAGCCCACA |
| | AAAGCAGTT-3′ | CCGTTCTC-3′ |
| OSCAR | 5′-ACACACACACC | 5′-GAGACCATCAAA |
| | TGGCACCTA-3′ | GGCAGAGC-3′ |
| GAPDH | 5′-TGCACCACCAA | 5′-GGATGCAGGGAT |
| | CTGCTTAG-3′ | GATGTTC-3′ |

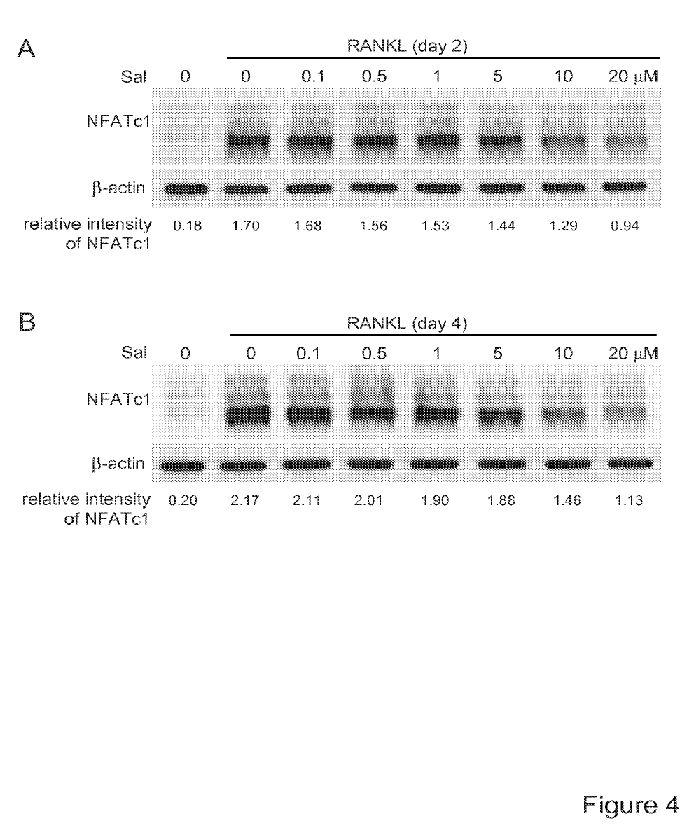

METHOD EXAMPLE. Downregulation of NFATcl in RAW264.7 cells by salubrinal. NFATcl is a transcription factor critical for activating osteoclastogenesis. Addition of RANKL to the culture medium significantly induced NFATcl expression day 2 and maintained its elevated level on day 4 (FIG. 4). The RANKL-induced expression of NFATcl was reduced by administration of 5-20 μM salubrinal on both days, and the effect of salubrinal was dose dependent (FIG. 4).

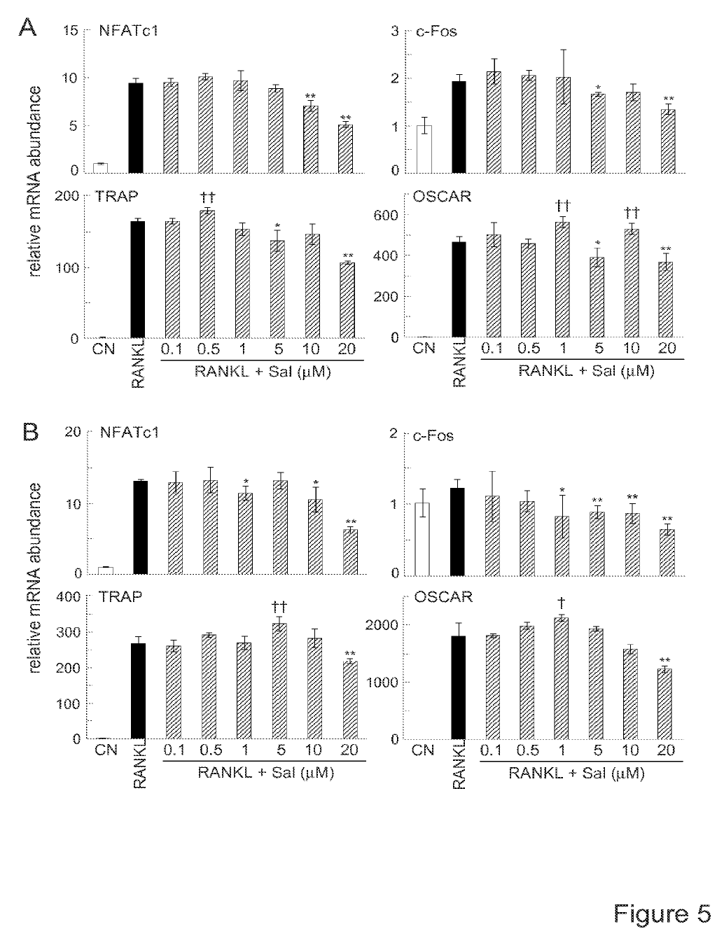

METHOD EXAMPLE. Partial suppression of mRNA levels of NFATcl, c-Fos, TRAP, and OSCAR by salubrinal. Addition of RANKL increased the mRNA levels of NFATcl, c-Fos, TRAP, and OSCAR, and administration of 20 μM salubrinal significantly reduced their mRNA levels. On day 2, for instance, the RANKL-driven increase was 9.4±0.5 fold (NFATcl), 1.9±0.1 fold (c-fos), 165±4.2 fold (TRAP), and 467±22 fold (OSCAR). The reduction by 20 μM salubrinal was 46% (NFATcl), 32% (c-fos), 35% (TRAP), and 21% (OSCAR) (FIG. 5A). Consistent with the observed dose response, administration of salubrinal at 0.1-1 μM did not contribute to significant reduction in these mRNA levels except for NFATcl and c-fos on day 4 (FIG. 5B).

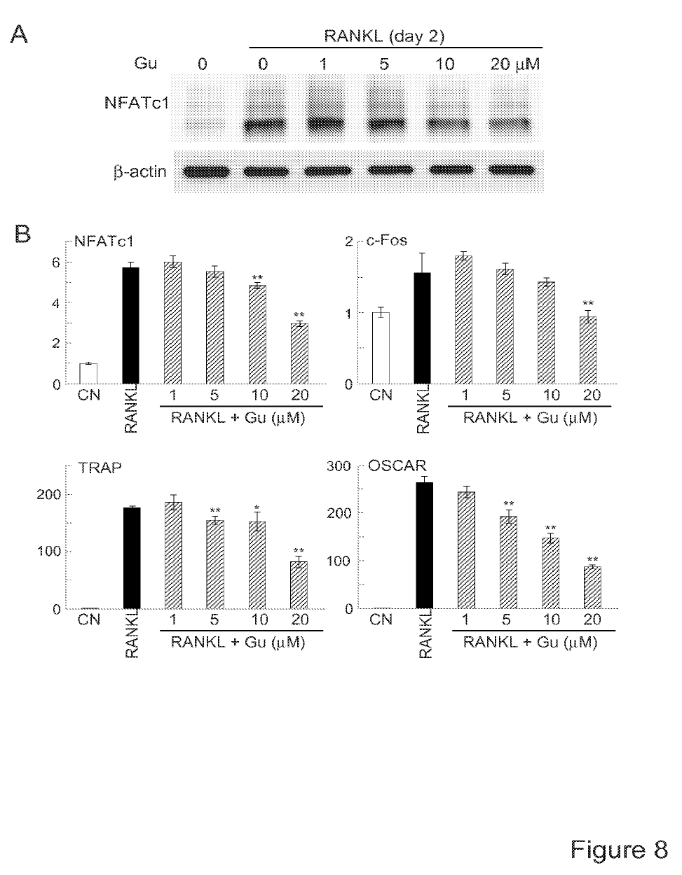

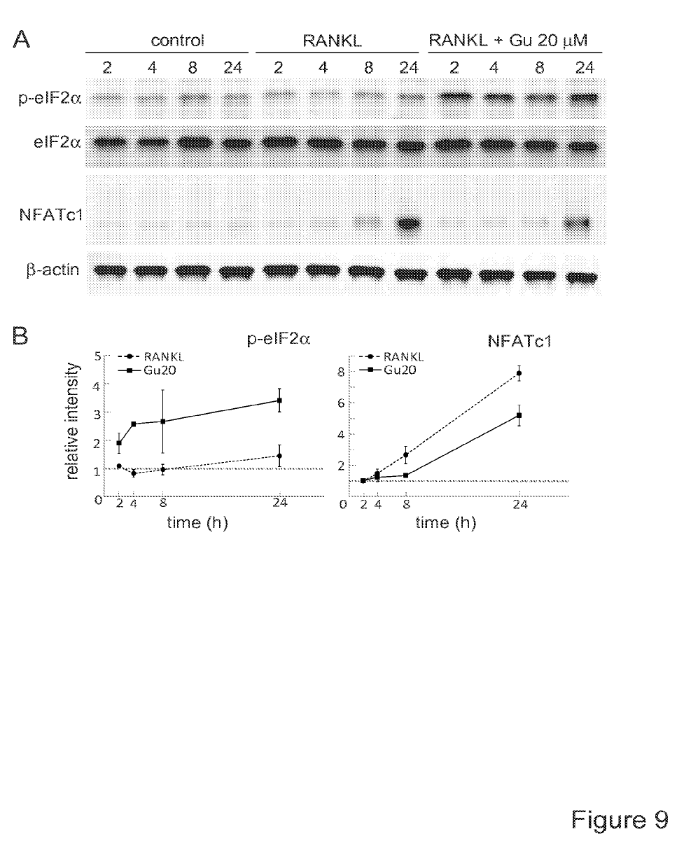

METHOD EXAMPLE. Reduction of RANKL-induced NFATcl, c-Fos, TRAP, and OSCAR by guanabenz. The induction of NFATcl by RANKL was suppressed by guanabenz in a dose dependent manner (FIG. 8A). The mRNA levels of NFATcl, c-Fos, TRAP, and OSCAR were also reduced by administration of 20 μM guanabenz. Lower concentrations of guanabenz, 5 and 10 μM, were effective in reducing the levels of TRAP and OSCAR mRNA (FIG. 8B). The temporal expression profile of p-eIF2α and NFATcl in response to 20 μM guanabenz revealed that p-eIF2α was upregulatcd in 2 h and NFATc 1 was partially suppressed in 8 h (FIG. 9). The normalized level of “1” was defined as the level for the cells that were not treated with RANKL without administration of guanabenz. In the absence of RANKL administration, however, either salubrinal or guanabenz did not significantly alter cell mortality and expression of NFATcl and TRAP (Supplementary FIG. S4).

METHOD EXAMPLE. Western Immunoblotting. Cells were lysed in a radioimmunoprecipitation assay (RIPA) buffer containing protease inhibitors (Santa Cruz Biotechnology, Santa Cruz, Calif., USA) and phosphatase inhibitors (Calbiochem, Billerica, Mass., USA). Isolated proteins were fractionated using 10-15% SDS gels and electro-transferred to Immobilon-P membranes (Millipore, Billerica, Mass., USA). The membrane was incubated for 1 h with primary antibodies followed by 45 min incubation with goat anti-rabbit or anti-mouse IgG conjugated with horseradish peroxidase (Cell Signaling, Danvers, Mass., USA). We used antibodies against ATF4, NFATcl (Santa Cruz), p-eIF2α (Thermo Scientific, Waltham, Mass., USA), eIF2α, caspase 3, cleaved caspase 3, p38 and p-p38 mitogen activated protein kinase (MAPK), extracellular signal-regulated kinase (ERK) and p-ERK, nuclear factor kappa B (NFκB) p65 and p-NFκB p65 (Cell Signaling), and β-actin (Sigma). Protein levels were assayed using a SuperSignal west femto maximum sensitivity substrate (Thermo Scientific), and signal intensities were quantified with a luminescent image analyzer (LAS-3000, Fuji Film, Tokyo, Japan).

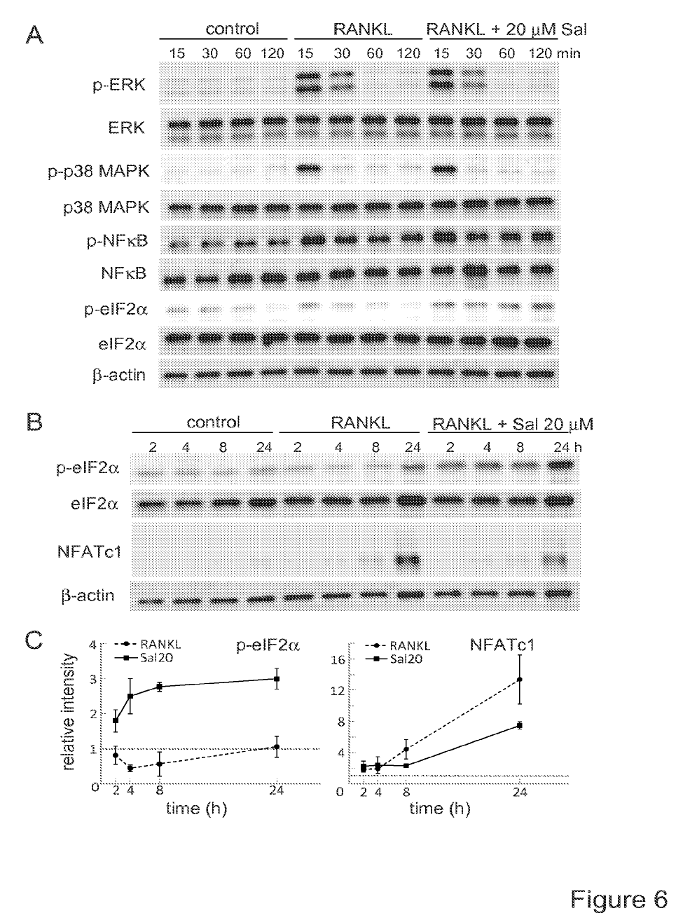

METHOD EXAMPLE. Temporal profile of p-eIF2α and NFATcl. The temporal expression profile revealed that addition of RANKL transiently reduced the phosphorylation level of eIF2α (2-8 h) and elevated NFATcl by 13.4±3.2 fold (24 h) (FIG. 6). This induction of NFATcl was partially suppressed by salubrinal with an increase in the level of p-eIF2α. In the early period (2-4 h), administration of 20 μM salubrinal increased the level of p-eIF2α but did not alter the level of NFATcl. In the later period (8-24 h), however, the level of NFATcl was significantly reduced by 48% (8 h) and 44% (24 h). Administration of 20 μM salubrinal did not significantly alter the phosphorylation level of ERK, p38 MAPK, and NFκB (FIG. 6). Note that the normalized level of “1” in FIG. 6C was defined as the level for the cells that were not treated with RANKL without administration of guanabenz.

METHOD EXAMPLE. Knockdown of ATF4 and eIF2α by siRNA. Cells were treated with siRNA specific to ATF4 and eIF2α (Life Technologies). Selected target sequences for knockdown of ATF4 and eIF2α were: ATF4, 5′-GCU GCU UAC AUU ACU CUA A-3′; and eIF2α, 5′-CGG UCA AAA UUC GAG CAG A-3′. As a nonspecific control, a negative siRNA (Silencer Select #1, Life Technologies) was used. Cells were transiently transfected with siRNA for ATF4, eIF2α or control in Opti-MEM I medium with Lipofectamine RNAiMAX (Life Technologies). Six hours later, the medium was replaced by regular culture medium. The efficiency of silencing was assessed with immunoblotting or quantitative PCR 48 h after transfection.

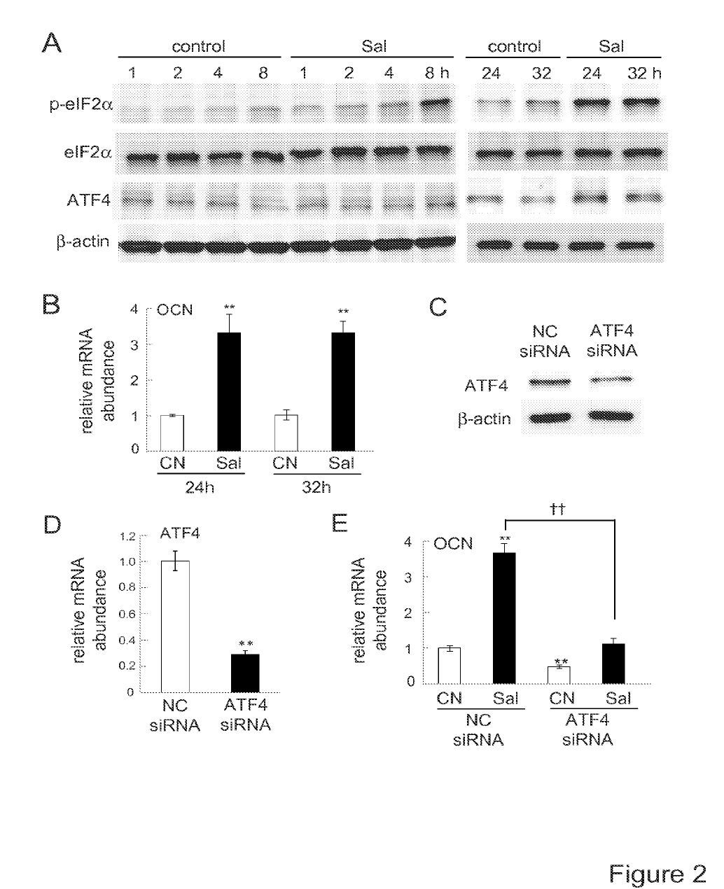

METHOD EXAMPLE. ATF4-mediated elevation of osteocalcin mRNA in MC3T3 E1-14 cells. Salubrinal is an inhibitor of de-phosphorylation of eIF2α. Administration of 5 μM salubrinal to MC3T3 E1-14 cells elevated phosphorylation of eIF2α, followed by an increase in ATF4 expression (FIG. 2A). Furthermore, the level of osteocalcin mRNA was increased 3.3±0.5 fold (24 h) and 3.3±0.3 fold (32 h) (FIG. 2B). When expression of ATF4 was significantly reduced by RNA interference (FIG. 2C & 2D), however, salubrinal-driven elevation of the osteocalcin mRNA level was suppressed (FIG. 2E). Non-clonal MC3T3 E1 cells also presented elevation of p-eIF2α and ATF4, together with an increase in the mRNA levels of ATF4 and osteocalcin (Supplementary FIG. S2). In addition, administration of guanabenz to MC3T3 E1-14 elevated the mRNA level of osteocalcin in a dose dependent manner, consistent with an increase in p-eIF2α and ATF4 (Supplementary FIG. S3).

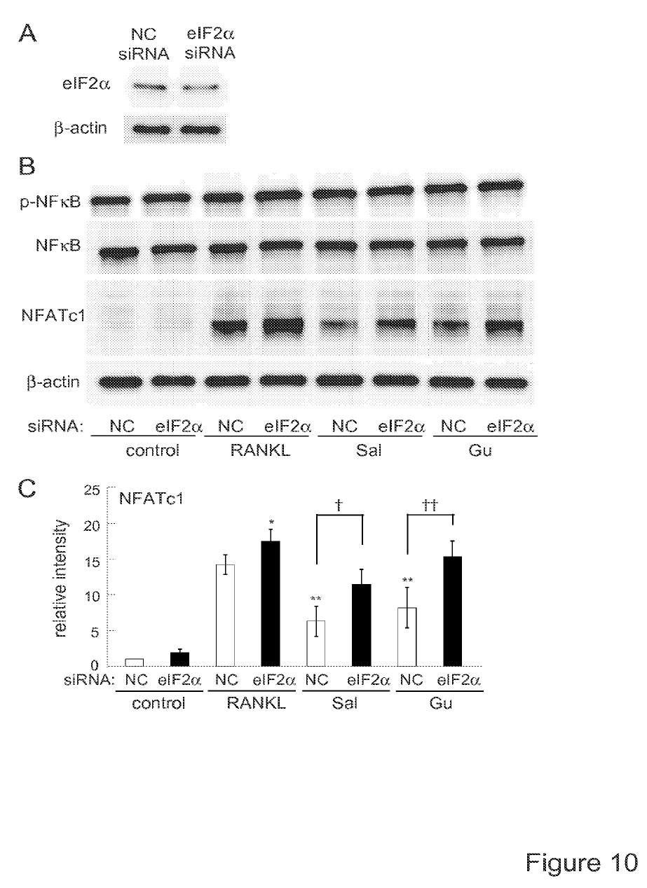

METHOD EXAMPLE. Reduction in salubrinal/guanabenz-driven suppression of NFATcl expression by RNA interference for eTF2α. To evaluate the effects of eIF2α on the expression level of NFATcl, RNA interference specific for eIF2α together with a non-specific control (NC) was used (FIG. 10). In response to 20 μM salubrinal, RAW264.7 cells transfected with the control siRNA demonstrated a reduction of NFATcl by 56%. However, the expression of NFATcl was reduced only by 20% in the cells transfected with eIF2α siRNA. Furthermore, 20 μM guanabenz decreased the level of NFATcl by 43% in the cells transfected with the control siRNA but the transfection of eIF2α siRNA abolished the suppressive effect of guanabenz. The phosphorylation level of NFκB was not significantly altered by transfection with eIF2α siRNA.

METHOD EXAMPLE. Mineralization assay. Mineralization of extracellular matrix was assayed by Alizarin Red S staining. MC3T3-E1 cells were plated in 6-well plates. When cells were confluent, 50 μg/ml of ascorbic acid (Wako Chemicals, Richmond, Va., USA) and 5 mM β-glycerophosphate (Sigma) were added. The medium was changed every other day, and staining was conducted after 3 week. Cells were washed with PBS twice and fixed with 60% isopropanol for 1 min at room temperature, followed by rehydration with distilled water for 3 min at room temperature. They were stained with 1% Alizarin red S (Sigma) for 3 min and washed with distilled water.

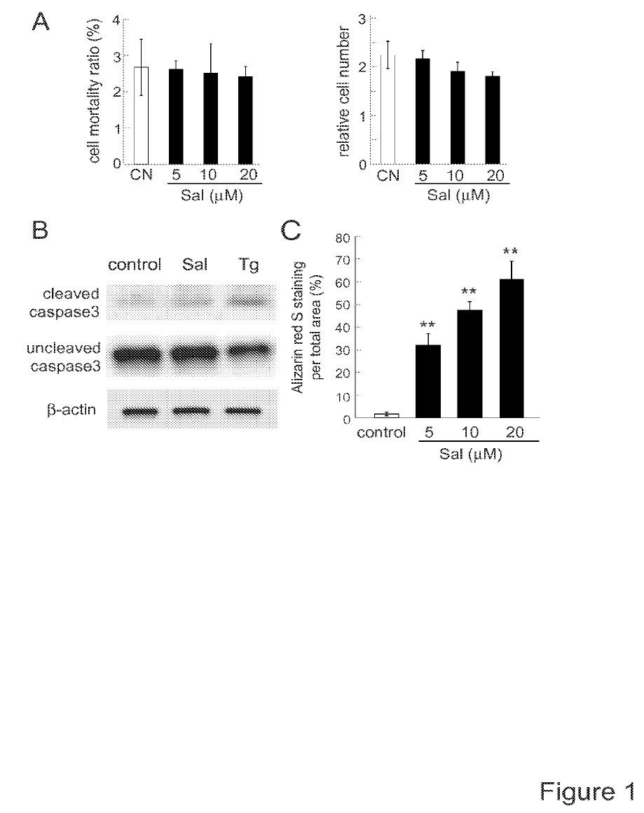

METHOD EXAMPLE. Enhanced mineralization of MC3T3 E1-14 cells by salubrinal. Prior to examining the effects of salubrinal on osteoclastogenesis, its effects on development of osteoblasts was tested, focusing on cell viability, phosphorylation of eIF2α (p-eIF2α), expression of ATF4 and osteocalcin, and matrix mineralization. Administration of 5-20 μM salubrinal to MC3T3 E1-14 cells did not increase cell mortality or inhibit cell proliferation (FIG. 1A). Unlike application of 10 nM thapsigargin, which is a stress inducer to the endoplasmic reticulum that elevates p-eIF2α, incubation with 10 μM salubrinal for 24 h did not elevate the expression level of cleaved caspase 3 (FIG. 1B). After 3-week incubation in an osteogenic medium, Alizarin red S staining area showed that salubrinal enhanced mineralization of MC3T3 E1-14 cells in a dose dependent manner (FIG. 1C). The enhanced mineralization was also observed in non-clonal MC3T3 E1 cells (Supplementary FIG. S1).

METHOD EXAMPLE. Osteoclastogenesis in vitro and TRAP (Tartrate-resistant acid phosphatase) staining. RAW264.7 cells were plated at a density of 5×10³/cm² into a 12-well or a 60 mm dish, and cultured with 20 ng/ml RANKL in the presence and absence of salubrinal or guanabenz. The culture medium was replaced every 2 days. After 5 days of culture, the cells were stained for TRAP staining using an acid phosphatase leukocyte kit (Sigma). The number of TRAP-positive cells containing three or more nuclei was determined

METHOD EXAMPLE. Statistical analysis. Three or four-independent experiments were conducted and data were expressed as mean±S.D. For comparison among multiple samples, ANOVA followed by post hoc tests was conducted. Statistical significance was evaluated at p<0.05. The single and double asterisks and daggers indicate p<0.05 and p<0.01. To determine intensities in immunoblotting and areas of Alizarin red S staining, images were scanned with Adobe Photoshop CS2 (Adobe Systems, San Jose, Calif., USA) and quantified using Image J.

The following publications, and each of the additional publications cited herein are incorporated herein by reference:

[1] Karsenty G, Wagner E F (2002) Reaching a genetic and molecular understanding of skeletal development. Cell 2: 389-406

[2] Harada S, Rodan G A (2003) Control of osteoblast function and regulation of bone mass. Nature 423: 349-355

[3] Manolagas S C (2000) Birth and death of bone cells: Basic regulatory mechanisms and implications for the pathogenesis and treatment of osteoporosis. Endocr Rev 21: 115-137.

[4] Kimball S C (1999) Eukaryotic initiation factor eIF2. Int J Biochem 31: 25-39

[5] Harding H P, Novoa I, Zhang Y, Zeng H, Wek R, Schapira M, Ron D (2000) Regulated translation initiation controls stress-induced gene expression in mammalian cells. Mol Cell 6: 1099-1108

[6] Yang X, Matsuda K, Bialek P, Jacquot S, Masuoka H C, Schinke T, Li L, Brancorsini S, Sassone-Corsi P, Townes T M, Hanauer A, Karsenty G (2004) ATF4 is a substrate of RSK2 and an essential regulator of osteoblast biology; implication for Coffin-Lowry Syndrome. Cell 117: 387-398

[7] Hamamura, K, Yokota H (2007) Stress to endoplasmic reticulum of mouse osteoblasts induces apoptosis and transcriptional activation for bone remodeling. FEBS Lett 581: 1769-1774

[8] Zhang P, Hamamura K, Jiang C, Zhao L, Yokota, H (2012) Salubrinal promotes healing of surgical wounds in rat femurs. J Bone Miner Metab 30: 568-579

[9] Yasuda H, Shima N, Nakagawa N, Yamaguchi K, Kinosaki M, Mochizuki S, Tomoyasu A, Yano K, Goto M, Murakami A, Tsuda E, Morinaga T, Higashino K, Udagawa N, Takahashi N, Suda T (1988) Osteoclast differentiation factor is a ligand for osteoprotegerin/osteoclastogenesis-inhibitory factor and is identical to TRANCE/RANKL. Proc Natl Acad Sci USA 95:3597-3602

[10] Lacey D L, Timms E, Tan H L, Kelley M J, Dunstan C R, Burgess T, Elliott R, Colombero A, Elliott G, Scully S, Hsu H, Sullivan J, Hawkins N, Davy E, Capparelli C, Eli A, Qian Y X, Kaufman S, Sarosi T, Shalhoub V, Senaldi G, Guo J, Delaney J, Boyle W J (1998) Osteoprotegerin ligand is a cytokine that regulates osteoclast differentiation and activation. Cell 93: 165-176

[11] Robling A G, Niziolek P J, Baldridge L A, Condon K W, Allen M R, Alam I, Mantila S M, Gluhak-Heinrich J, Bellido T M, Harris S E, Turner C H (2008) Mechanical stimulation of bone in vivo reduces osteocyte expression of Sost/sclerostin. J Biol Chem 283: 5866-5875

[12] Zhang P, Jiang C, Ledet E, Yokota H (2011) Loading- and unloading-driven regulation of phosphorylation of eIF2 . Biol Sci Space 25: 3-6

[13] Boyce M, Bryant K F, Jousse C, Long K, Harding H P, Scheuner D, Kaufman R J, Ma D, Coen D M, Ron D, Yuan J (2005) A selective inhibitor of eIF2 dephosphorylation protects cells from ER stress. Science 307: 935-939

[14] Tsaytler P, Harding H P, Ron D, Bertolotti A (2011) Selective inhibition of a regulatory subunit of protein phosphatase 1 restores proteostasis. Science 332: 91-94

[15] Hughes D E, Wright K R, Uy H L, Sasaki A, Yoneda T, Roodman G D, Mundy G R, Boyce B F (1995) Bisphosphonates promote apoptosis in murine osteoclasts in vitro and in vivo. J Bone Miner Res 10: 1478-1487

[16] Furuya Y, Mori K, Ninomiya T, Tomimori Y, Tanaka S, Takahashi N, Udagawa N, Uchida K, Yasuda H (2011) Increased bone mass in mice after single injection of anti-receptor activator of nuclear factor—B ligand-neutralizing antibody. J Biol Chem 286: 37023-37031

[17] Sudo H, Kodama H A, Amagai Y, Yamamoto S, Kasai S (1983) In vitro differentiation and calcification in a new clonal osteogenic cell line derived from newborn mouse calvaria. J Cell Biol 96: 191-198

[18] Xu J, Tan J W, Gao X H, Laird R, Liu D, Wysocki S, Zheng M H (2000) Cloning, sequencing, and functional characterization of the rat homologue of receptor activator of NF-kappaB ligand. J Bone Miner Res 15: 2178-2186

[19] Wagner E F, Matsuo K (2003) Signaling in osteoclasts and the role of Fos/AP1 proteins. Ann Rheum Dis 62: ii83-ii85

[20] Helfrich M H, Thesingh C W, Mieremet R H, van Iperen-van Gent A S (1987) Osteoclast generation from human fetal bone marrow in cocultures with murine fetal long bones. A model for in vitro study of human osteoclast formation and function. Cell Tissue Res 249: 125-136

[21] Kim N, Takami M, Rho J, Josien R, Choi Y (2002) A novel member of the leukocyte receptor complex regulates osteoclast differentiation. J Exp Med 195: 201-209

[22] Takayanagi H, Kim S, Koga T, Nishina H, Isshiki M, Yoshida H, Saiura A, Isobe M, Yokochi T, Inoue J, Wagner E F, Mak T W, Kodama T, Taniguchi T (2002) Induction and activation of the transcription factor NFATcl (NFAT2) integrate RANKL signaling in terminal differentiation of osteoclasts. Dev Cell 3: 889-901

[23] Hamamura K, Zhang P, Yokota H (2008) IGF2-driven PI3 kinase and TGF signaling pathways in chondrogenesis. Cell Biol Int 32: 1238-1246

[24] Shah R S, Walker B R, Vanov S K, Helfant R H (1976) Guanabenz effects on blood pressure and noninvasive parametors of cardiac performance in patients with hypertension. Clin Pharmacol Ther 19: 732-737

[25] Galli M, Caniggia M (1984) Osteocalcin. Minerva Med 75: 2489-2501

[26] Angel N Z, Walsh N, Forwood, M R, Ostrowski M C, Cassady A I, Hume D A (2000) Transgenic mice overexpressing tartrate-resistant acid phosphatase exhibit an increase rate of bone turnover. J Bone Miner Res 15: 103-110

[27] Wang S, Kaufman R J (2012) The impact of the unfolded protein response on human disease. J Cell Biol 197: 857-867

[28] Matsumoto M, Sudo T, Saito T, Osada H, Tsujimoto M (2000) Involvement of p38 mitogen-activated protein kinase signaling pathway in osteoclastogenesis mediated by receptor activator of NF-kappaB ligand (RANKL). J Biol Chem 275: 31155-31161

[29] Zhang Y H, Heulsmann A, Tondravi M M, Mukherjee A, Abu-Amer Y (2001) Tumor necrosis factor-alpha (TNF) stimulates RANKL-induced osteoclastogenesis via coupling of TNF type 1 receptor and RANK signaling pathways. J Biol Chem 276: 563-568

[30] Kim K, Kim J H, Lee J, Jin H M, Kook H, Kim K K, Lee S Y, Kim N (2007) MafB negatively regulates RANKL-mediated osteoclast differentiation. Blood 109:3253-3259

[31] Zhao B, Takami M, Yamada A, Wang X, Koga T, Hu X, Tamura T, Ozato K, Choi Y, Ivashkiv L B, Takayanagi H, Kamijo R (2009) Interferon regulatory factor-8 regulates bone metabolism by suppressing osteoclastogenesis. Nat Med 15:1066-1071

[32] Miyauchi Y, Ninomiya K, Miyamoto H, Sakamoto A, Iwasaki R, Hoshi H, Miyamoto K, Hao W, Yoshida S, Morioka H, Chiba K, Kato S, Tokuhisa T, Saitou M, Toyama Y, Suda T Miyamoto T (2010). The Blimp1-Bcl6 axis is critical to regulate osteoclast differentiation and bone homeostasis. J Exp Med 207:751-762

[33] Rossi M, Pitari M R, Amodio N, Di Martino M T, Conforti F, Leone E, Botta C, Paolino F M, Del Giudicc T, Iuliano E, Caraglia M, Ferrarini M, Giordano A, Tagliaferri P, Tassone P (2012) miR-29b negatively regulates human osteoclastic cell differentiation and function: Implications for the treatment of multiple myeloma-related bone disease. J Cell Physiol in press

[34] Yasui T, Hirose S, Tsutsumi S, Nakamura K, Aburatani H, Tanaka S (2011) Epigenetic regulation of osteoclast differentiation: Possible involvement of Jmjd3 in the histone demethylation of Nfatcl. J Bone Miner Res 26: 2655-2671

[35] He L, Lee J, Jang J H, Sakchaisri K, Hwang, J S, Cha-Molstad H J, Kim K A, Ryoo I J, Lee H G, Kim S O, Soung N K, Lee K S, Kwon Y T, Erikson R L, Ahn J S, Kim B Y (2013) Osteoporosis regulation by salubrinal through eIF2 mediated differentiation of osteoclast and osteoblast. Cell Signal 25:552-560

What is claimed is:

1. A method for treating osteoporosis, fracture, or a bone defect in a host animal, the method comprising the step of administering to the host animal a therapeutically effective amount of one or more compounds capable of selectively inhibiting de-phosphorylation of eIF2α; where the amount is capable of (a) stimulating matrix deposition of osteoblasts, (b) upregulating activating transcription factor 4 (ATF4), (c) reducing expression of nuclear factor of activated T cells cl (NFATcl), (d) inhibiting differentiation of RAW264.7 cells to multi-nucleated osteoclasts, (e) stimulating osteoblastogenesis, or (f) inhibiting osteoclastogenesis, or a combination of the foregoing, or a pharmaceutically acceptable composition comprising the one or more compounds.

2. The method claim 1 wherein at least one compound is of the formula 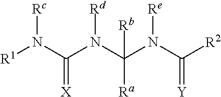
or a pharmaceutically acceptable salt thereof, wherein:
X is O or S;
Y is O or S;
R¹ is alkyl, cycloalkyl, alkenyl, cycloalkenyl, aryl, or heteroaryl, each of which is optionally substituted;
R² is alkyl, cycloalkyl, alkenyl, cycloalkenyl, aryl, heteroaryl, cycloalkylalkyl, cycloalkenylalkyl, arylalkyl, heteroarylalkyl, cycloalkylalkenyl, cycloalkenylalkenyl, arylalkenyl, or heteroarylalkenyl, each of which is optionally substituted;
R^{a} is optionally substituted alkyl;
R^{b} is H or optionally substituted C₁-C₆ alkyl;
R^{c}, R^{d}, and R^{e} are each independently selected from the group consisting of H, optionally substituted C₁-C₆ alkyl, acyl, or a prodrug capable of releasing the attached nitrogen in vivo.

3. The method claim 1 or 2 wherein at least one compound is of the formula 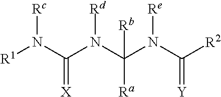
or a pharmaceutically acceptable salt thereof, wherein:
X is O or S;
Y is O or S;
R¹ is alkyl, cycloalkyl, alkenyl, cycloalkenyl, aryl, or heteroaryl, each of which is optionally substituted;
R² is alkyl, cycloalkyl, alkenyl, cycloalkenyl, aryl, heteroaryl, cycloalkylalkyl, cycloalkenylalkyl, arylalkyl, heteroarylalkyl, cycloalkylalkenyl, cycloalkenylalkenyl, arylalkenyl, or heteroarylalkenyl, each of which is optionally substituted;
R^{a} is optionally substituted alkyl;
R^{b} is H or optionally substituted C₁-C₆ alkyl;
R^{c}, R^{d}, and R^{e} are each independently selected from the group consisting of H, optionally substituted C₁-C₆ alkyl, acyl, or a prodrug capable of releasing the attached nitrogen in vivo;
or a composition thereof further comprising one or more carriers, diluents, or excipients, or a combination thereof.

4. The method of any one of the preceding claims wherein at least one compound is of the formula 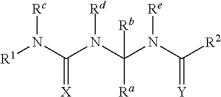
or a pharmaceutically acceptable salt thereof, wherein:
X is O or S;
Y is O or S;
R¹ is alkyl, cycloalkyl, alkenyl, cycloalkenyl, aryl, or heteroaryl, each of which is optionally substituted;
R² is alkyl, cycloalkyl, alkenyl, cycloalkenyl, aryl, heteroaryl, cycloalkylalkyl, cycloalkenylalkyl, arylalkyl, heteroarylalkyl, cycloalkylalkenyl, cycloalkenylalkenyl, arylalkenyl, or heteroarylalkenyl, each of which is optionally substituted;
R^{a} is optionally substituted alkyl;
R^{b} is H or optionally substituted C₁-C₆ alkyl;
R^{c}, R^{d}, and R^{e} are each independently selected from the group consisting of H, optionally substituted C₁-C₆ alkyl, acyl, or a prodrug capable of releasing the attached nitrogen in vivo.

5. The method of any one of the preceding claims wherein R¹ is alkyl, aryl, or heteroaryl, each of which is optionally substituted.

6. The method of any one of the preceding claims wherein R¹ is aryl or heteroaryl, each of which is optionally substituted.

7. The method of any one of the preceding claims wherein R² is alkenyl, heteroaryl, cycloalkylalkyl, cycloalkenylalkyl, arylalkyl, heteroarylalkyl, cycloalkylalkenyl, cycloalkenylalkenyl, arylalkenyl, or heteroarylalkenyl, each of which is optionally substituted.

8. The method of any one of the preceding claims wherein R² is alkenyl, cycloalkylalkyl, cycloalkenylalkyl, arylalkyl, heteroarylalkyl, cycloalkylalkenyl, cycloalkenylalkenyl, arylalkenyl, or heteroarylalkenyl, each of which is optionally substituted.

9. The method of any one of the preceding claims wherein at least one compound is a compound of the formula 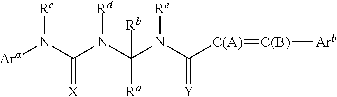
or a pharmaceutically acceptable salt thereof, wherein:
X and Y are independently O or S;
Ar^{a} and Ar^{b} are independently aryl or heteroaryl, each of which is optionally substituted;
R^{a} is optionally substituted alkyl;
R^{b} is H or optionally substituted C₁-C₆ alkyl;
R^{c}, R^{d}, and R^{e} are each independently selected from the group consisting of H, optionally substituted C₁-C₆ alkyl, acyl, and a prodrug capable of releasing the attached nitrogen in vivo to form the corresponding H or salt derivative thereof;
and A and B are independently H, or optionally substituted C₁-C₆ alkyl

10. The method of claim 9 wherein the aryl is a bicyclic aryl.

11. The method of claim 9 or 10 wherein the heteroaryl is a bicyclic heteroaryl.

12. The method of any one of claims 9 to 11 wherein the aryl or heteroaryl is optionally substituted with one or more substituents selected from the group consisting of halo, nitrile, or optionally substituted C₁-C₆ alkyl, C₁-C₆ alkoxy, C₁-C₆ haloalkyl, C₁-C₆ haloalkoxy, aryl, aryloxy, heterocyclyl, heterocyclyloxy, fused aryl, and fused heterocyclyl.

13. The method of any one of claims 9 to 12 wherein the alkenyl has an E geometry.

14. The method of any one of the preceding claims wherein at least one compound is of the formula 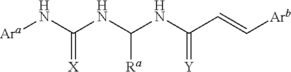
or a pharmaceutically acceptable salt thereof, wherein,
X is O or S;
Y is O or S;
Ar^{a} and Ar^{b} are independently aryl or heteroaryl, each of which is optionally substituted; and
R^{a} is optionally substituted alkyl.

15. The method of claim 14 wherein the aryl is a bicyclic aryl.

16. The method of claim 14 or 15 wherein Ar^{a} is a bicyclic heteroaryl.

17. The method of any one of claims 14 to 16 wherein the aryl or heteroaryl is optionally substituted with one or more substituents selected from the group consisting of halo, nitrile, or optionally substituted C₁-C₆ alkyl, C₁-C₆ alkoxy, C₁-C₆ haloalkyl, C₁-C₆ haloalkoxy, aryl, aryloxy, heterocyclyl, heterocyclyloxy, fused aryl, and fused heterocyclyl.

18. The method of any one of claims 14 to 17 wherein R^{a} is haloalkyl.

19. The method of any one of claims 14 to 18 wherein R^{a} is not trifluoromethyl.

20. The method of any one of claims 14 to 19 wherein R^{a} is haloalkyl, where halo is selected from the group consisting of chloro and bromo, and combinations thereof.

21. The method of any one of claims 14 to 20 wherein R^{c}, R^{d}, and R^{e} are each independently selected from the group consisting of H, optionally substituted C₁-C₆ alkyl, and acyl.

22. The method of any one of the preceding claims wherein at least one compound is salubrinal, or an analog or a derivative thereof, or a pharmaceutically acceptable salt of the foregoing.

23. The method of any one of the preceding claims wherein at least one compound is of the formula 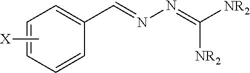
or a pharmaceutically acceptable salt thereof, wherein
X represents five substituents each independently selected from hydrogen, halo, hydroxy and derivatives thereof, amino and derivatives thereof, thio and derivatives thereof, carboxylate or a derivative thereof, sulfinyl or a derivative thereof, sulfonyl or a derivative thereof, phosphinyl or a derivative thereof, or phosphonyl or a derivative thereof, or alkyl, alkenyl, alkynyl, cycloalkyl, cycloalkenyl, heteroalkyl, heteroalkenyl, cycloheteroalkyl, cycloheteroalkenyl, aryl, heteroaryl, arylalkyl, or heteroarylalkyl, each of which is optionally substituted; and
R is independently selected in each instance from hydrogen, hydroxy and derivatives thereof, amino and derivatives thereof, carboxylate and derivatives thereof, sulfonyl and derivatives thereof, and alkyl, alkenyl, alkynyl, cycloalkyl, cycloalkenyl, heteroalkyl, heteroalkenyl, cycloheteroalkyl, cycloheteroalkenyl, aryl, heteroaryl, arylalkyl, and heteroarylalkyl, each of which is optionally substituted, and nitrogen prodrug forming groups.

24. The method of claim 23 wherein at least one X is halo, such as chloro.

25. The method of claim 23 wherein at least one X is optionally substituted alkyl, such as methyl or trifluoromethyl.

26. The method of claim 23 wherein at least one X is optionally substituted alkoxy, such as methoxy or trifluoromethoxy.

27. The method of any one of the preceding claims wherein at least one compound is of the formula 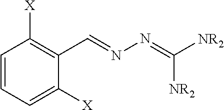
or a pharmaceutically acceptable salt thereof

28. The method of claim 27 wherein each X is halo.

29. The method of claim 27 wherein each X is chloro.

30. The method of any one of claims 27 to 29 wherein each R is hydrogen

31. The method of any one of claims 27 to 30 wherein at least one compound is guanabenz or a pharmaceutically acceptable salt thereof.

32. The method of any one of the preceding claims wherein the composition further comprises one or more carriers, diluents, or excipients, or a combination thereof.

33. The method of any one of the preceding claims wherein the host animal is a human.
